(19) United States
(12) Patent Application Publication
(10) Pub. No.: US 2026/0206703 A1
(43) Pub. Date: Jul. 23, 2026

(54) REGENERATION BY PROTOPLAST CALLUS GRAFTING

(71) Applicant: KEYGENE N.V., Wageningen (NL)

(72) Inventors: Hugo Ferdinand HOFHUIS, Wageningen (NL); Jeroen STUURMAN, Wageningen (NL); Bipna Rani SHRESTHA, Wageningen (NL)

(73) Assignee: KEYGENE N.V., Wageningen (NL)

(21) Appl. No.: 19/135,838

(22) PCT Filed: Dec. 22, 2023
(86) PCT No.: PCT/EP2023/087536

§ 371 Date: Jun. 5, 2025

(30) Foreign Application Priority Data
Dec. 22, 2022 (EP) 22215800.8

(51) Int. Cl. A01H 4/00

(52) U.S. Cl. CPC A01H 4/008

(57) ABSTRACT

The invention concerns a method for producing a shoot of a plant comprising germline progenitor cells of a recalcitrant plant. The germline progenitor cells may be modified to comprise a mutation in a sequence of interest. The invention further pertains to plants obtainable by the method of the invention, wherein the plant preferably comprises at least the L2-meristem layer of the recalcitrant plant.

### FIELD OF THE INVENTION

The present invention relates to the field of molecular plant biology, in particular to the field of plant regeneration. The invention concerns methods for improving the regeneration capacity and/or regeneration efficiency of plants.

### BACKGROUND

As in general plant cells have a limited capacity to regenerate, regeneration from a single cell to a whole plant is a bottleneck in many plant biotechnology workflows. Such workflows encompass protocols for targeted genome editing, clonal propagation of haploid or genetically complex plant material, production of stable transformants and induction of doubled haploids. Although regeneration capacity varies between different plant species, plant variety and plant tissue origin, the fraction of cells successfully regenerating to plants is usually quite low (Srinivasan et al., Planta 2007, 225:341-351). Plant species or varieties in which the regeneration fails or the efficiency is poor are considered recalcitrant.

As many biotechnology workflows, for instance genome editing by programmed nucleases based on DNA-free protocols, involve protoplasts, the major hurdle of these rapidly evolving technologies is the regeneration of whole plants from these single (edited) cells. There is thus a strong need in the art to increase the regeneration efficiency of a plant, preferably to increase the regeneration efficiency of a recalcitrant plant. In particular, there is a strong need for increasing the regeneration efficiency of a plant carrying a heritable mutation.

### SUMMARY

The invention may be summarized in the following embodiments:
Embodiment 1. Method of generating and selecting a shoot of a plant, wherein the method comprises the steps of:
   (a) intergrafting a callus between a scion and a rootstock;
   (b) allowing graft junctions to be formed between the callus and each one of the scion and the rootstock, to form a grafted union;
   (c) generating a wound at or near at least one of the graft junctions;
   (d) allowing the wounded grafted union to form shoots;
   (e) selecting a shoot formed in step (d), wherein said shoot comprises cells derived from the callus of step (a); and optionally
   (f) growing a plant from the selected shoot of step (e).
Embodiment 2. Method according to embodiment 1, wherein in step (e) the selected shoot comprises a germline progenitor cell derived from the callus of step (a) and wherein optionally the method further comprises step (f) and a step (g) of obtaining seed or progeny of the plant grown in step (f), preferably by sexual propagation, wherein the sexual propagation is preferably at least one of selfing and backcrossing.
Embodiment 3. Method according to embodiment 1 or 2, wherein the method further comprises step (f) and a step (g) of obtaining progeny of the plant grown in step (f) by vegetative propagation.
Embodiment 4. Method according to any one of embodiments 1-3, wherein the callus in step (a) is of a first plant and the scion and/or rootstock are of a second plant.
Embodiment 5. Method according to embodiment 4, wherein the scion and the rootstock are of the same or a similar plant.
Embodiment 6. Method according to any one of the preceding embodiments, wherein the wounding of step (c) is removal of the shoot apical meristem by decapitation.
Embodiment 7. Method according to any one of the preceding embodiments, wherein in step (d) the axis of the (wounded) grafted union is substantially perpendicular to the earth surface, and wherein the root apical meristem is closer to the earth surface as compared to the shoot apical meristem.
Embodiment 8. Method according to any one of the preceding embodiments, wherein step (d) of allowing shoot formation comprises the steps of:
   d1) allowing callus to be formed at or near the graft junction; and
   d2) allowing a shoot to grow from said callus.
Embodiment 9. Method according to any one of the preceding embodiments, wherein the method further comprises prior to step (a) a step of growing the callus of step (a) from a protoplast.
Embodiment 10. Method according to embodiment 9, wherein the method further comprises a step of introducing into the protoplast a transgene and/or a mutation in a sequence of interest, and wherein in step (e) the selected shoot comprises a germline progenitor cell, or a germline cell derived therefrom, comprising the transgene and/or the mutation.
Embodiment 11. Method according to any one of the preceding embodiments, wherein the method further comprises a step of introducing into a cell located in the callus of step (a) and/or in the shoot formed in step (d) a transgene and/or a mutation in a sequence of interest, and wherein in step (e) the selected shoot comprises a germline progenitor cell, or a germline cell derived therefrom, comprising the transgene and/or the mutation.
Embodiment 12. Method according to embodiment 10 or 11, wherein the method comprises step (f) and wherein a plant part of the plant grown in step (f) comprises the transgene and/or the mutation, and wherein preferably the plant part can be used for vegetative propagation.
Embodiment 13. Method according to any one of embodiments 10-12, wherein the mutation is introduced by programmed genome editing, preferably using a site-specific endonuclease, preferably a CRISPR endonuclease.
Embodiment 14. Plant obtainable by the method of any one of embodiments 10-13, wherein said plant comprises at least one of:
   i) a germline progenitor cell and/or a germline cell derived therefrom, of the callus of step (a); and
   ii) a plant part for vegetative propagation of the callus of step (a),
wherein the germline progenitor cell, germline cell and/or a plant part comprises the transgene and/or the mutation of embodiment 10 or 11.
Embodiment 15. Plant according to embodiment 14, wherein said plant comprises cells derived from the callus of step (a) and cells derived from the scion and/or rootstock of step (a).

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

It is clear for the skilled person that any methods and materials similar or equivalent to those described herein can be used for practising the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

The singular terms “a,” “an,” and “the” include plural referents unless the context clearly dictates otherwise. Thus, for example, reference to “a cell” includes a combination of two or more cells, and the like. The indefinite article “a” or “an” thus usually means “at least one”.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ± (+ or −) 10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

“Plant” refers to either the whole plant or to parts of a plant tissue or organs (e.g. pollen, seeds, roots, leaves, flowers, flower buds, anthers, fruit, etc.) obtainable from the plant, as well as derivatives of any of these and progeny derived from such a plant by selfing or crossing or apomictic reproduction. Non-limiting examples of plants include crop plants and cultivated plants, such as Affrican eggplant, alliums, artichoke, asparagus, barley, beet, bell pepper, bitter gourd, bladder cherry, bottle gourd, cabbage, canola, carrot, cassava, cauliflower, celery, chicory, common bean, corn salad, cotton, cucumber, eggplant, endive, fennel, gherkin, grape, hot pepper, lettuce, maize, melon, oilseed rape, okra, parsley, parsnip, pepino, pepper, potato, pumpkin, radish, rice, ridge gourd, rocket, rye, snake gourd, sorghum, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism, from plant origin. The plant cell can e.g. be part of a multicellular structure, such as a callus, meristem, plant organ or an explant. A plant cell may be a meristematic cell, a somatic cell and/or a reproductive cell.

A “scion” is a shoot of a plant, preferably a young shoot preferably of a young plant, cut for grafting. A scion preferably has a functional shoot apical meristem.

A “rootstock” is a stem of a plant, preferably of a young plant, with a functional root system preferably comprising a functional root apical meristem.

“Similar conditions” for culturing the plant/plant cells means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). gap extension penalty is 0.5; default scoring matrices are Blossum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Alternatively percentage similarity or identity may be determined by searching against public databases, using algorithms such as FASTA, BLAST, etc. Thus, the nucleic acid and protein sequences described herein can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules described herein. BLAST protein searches can be performed with the BLASTx program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “nucleic acid” or “polynucleotide” as used herein may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). Contemplated are any deoxyribonucleotide, ribonucleotide or nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA (optionally cDNA) or RNA, or a mixture thereof, and may exist permanently or transitionally in single-stranded or double-stranded form, including homoduplex, heteroduplex, and hybrid states.

An “isolated nucleic acid” is used to refer to a nucleic acid which is no longer in its natural environment, for example in vitro or in a recombinant bacterial or plant cell. A nucleic acid and/or protein may be at least one of a recombinant, synthetic or artificial nucleic acid and/or protein.

The terms “nucleic acid construct”, “nucleic acid vector”, “vector” and “expression construct” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules.

The vector backbone may for example be a binary or superbinary vector (see e.g. U.S. Pat. No. 5,591,616, US2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, which is biologically active, e.g. which is capable of being translated into a biologically active protein or peptide, or e.g. a regulatory non-coding RNA.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation).

A “3′ UTR” or “3′ non-translated sequence” (also often referred to as 3′ untranslated region, or 3′ end) refers to the nucleic acid sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

The term “cDNA” means complementary DNA. Complementary DNA is made by reverse transcribing RNA into a complementary DNA sequence. cDNA sequences thus correspond to RNA sequences that are expressed from genes. As RNA sequences expressed from the genome can undergo splicing, i.e. introns are spliced out of the pre-mRNA and exons are joined together, before being translated in the cytoplasm into proteins, it is understood that expression of a cDNA means expression of the mRNA that encodes for the cDNA. The cDNA sequence thus may not be identical to the genomic DNA sequence to which it corresponds as the cDNA may encode only the complete open reading frame, consisting of the joined exons, for a protein, whereas the genomic DNA sequence may comprise exon sequences interspersed by intron sequences. Genetically modifying a gene which encodes a protein may thus not only relate to modifying the sequences encoding the protein, but may also involve mutating intronic sequences of the genomic DNA and/or other gene regulatory sequences of that gene.

The term “regeneration” is herein defined as the formation of a new tissue and/or a new organ from a single plant cell, a group of cells, a callus, an explant, a tissue or from an organ. Regeneration may include the formation of a new plant from a single plant cell or from e.g. a callus, an explant, a tissue or an organ. The plant cell for regeneration can be an undifferentiated plant cell. A preferred plant cell is a protoplast. The regeneration process can occur directly from parental tissues or indirectly, e.g. via the formation of a callus. The regeneration pathway can be somatic embryogenesis or organogenesis. Somatic embryogenesis is understood herein as the formation of somatic embryos, which can be grown into whole plants. Organogenesis is understood herein as the formation of new organs from (undifferentiated) cells. Organogenesis may be at least one of meristem formation, adventitious shoot formation, inflorescence formation, root formation, elongation of adventitious shoots and (subsequent) the formation of a complete plant. Preferably, regeneration is at least one of shoot regeneration, (ectopic) apical meristem formation and root regeneration. Shoot regeneration as defined herein is de novo shoot formation. For example, regeneration can be the regeneration of a (n) (inflorescence) shoot from a (n) (elongated) hypocotyl explant.

The term “normal growth conditions” is herein understood as an environment wherein a plant grows. Such conditions include at minimum a suitable temperature (i.e. between 0° C.-60° C.), nutrition, day/night rhythm and irrigation.

The term “conditions that allow for regeneration” is herein understood as an environment wherein a plant cell or tissue can regenerate, preferably including normal growth conditions.

“Shoot organogenesis” is the regeneration pathway by which cells, preferably cells of callus or explant, form a de novo shoot apical meristem that develops into a shoot with leaf primordia and leaves. As there is only one apical meristem, this is a unipolar structure, and roots are not formed at this stage. The vascular system of the shoot is often connected to the parent tissue. Only after the shoots have fully formed and elongated, and are taken off e.g. the callus or explant, can the formation of roots be induced in a separate root induction step on a different culture medium (Thorpe, T A (1993) In vitro Organogenesis and Somatic Embryogenesis: Physiological and Biochemical Aspects. In: Roubelakis-Angelakis K. A., Van Thanh K. T. (eds) Morphogenesis in Plants. NATO ASI Series (Series A: Life Sciences), Vol. 253. Springer, Boston, MA).

Shoot organogenesis may occur spontaneously, i.e. without the external addition of any plant growth regulators (PGRs). Shoot organogenesis may be induced by plant growth regulators, usually cytokinins alone in different concentrations or in combination with an auxin, wherein preferably the cytokinins remain a constituent of the culture media until the new shoot apical meristems and the shoots have been formed and are sufficiently elongated, e.g. to take them off the primary explant or callus. Preferably, for the induction of shoot formation, the concentration of cytokinins exceeds the concentration of auxins.

“Somatic embryogenesis” leads to the formation of bipolar structures resembling zygotic embryos, which contain a root-shoot axis with a closed independent vascular system. In other words, both root and shoot primordia are being formed simultaneously, and there is no vascular connection to the underlying tissue (Dodds, J H and Roberts, L W (1985) Experiments in plant tissue culture. Cambridge University Press, Cambridge, UK). Somatic embryogenesis can e.g. be induced indirectly from callus or cell suspensions, or they can be induced directly on cells of explants (Thorpe, supra). Somatic embryo formation passes through a number of distinct stages, from globular stage (small isodiametric cell clusters), via heart stage (bilaterally symmetrical structures) to torpedo stage (elongation). The globular-to-heart transition is marked by the outgrowth of the two cotyledons and the beginning of the development of the radicle (Zimmerman, J L (1993) Somatic Embryogenesis: A Model for Early Development in Higher Plants. The Plant Cell 5:1411-1423; Von Arnold et al (2002) Developmental pathways of somatic embryogenesis. Plant Cell, Tissue and Organ Culture 69:233-249). Finally, torpedo-stage somatic embryos can develop into plantlets that contain green cotyledons, elongated hypocotyls, and developed radicles with clearly differentiated root hairs (Zimmerman, supra), in a process that is termed ‘germination’ (analogous to zygotic embryos) or ‘conversion’ or ‘maturation’ (Von Arnold et al., supra). In the induction of somatic embryogenesis, directly or indirectly, preferably auxins are used at the initial stage to induce an embryogenic state in the callus, but the embryos form after passage of the culture to a medium without or with reduced auxin levels. Auxins used for somatic embryo induction are e.g. 1-naphthaleneacetic acid (NAA), 2,4-dichlorophenoxyacetic acid (2,4-D), picloram and dicamba.

The term “endogenous” as used in the context of the present invention in combination with a protein or nucleic acid means that said protein or nucleic acid is still contained within the plant, i.e. is present in its natural environment. Often an endogenous gene will be present in its normal genetic context in the plant.

“Plant hormones”, “plant growth hormone”, “plant growth regulator” or “phytohormone” is a chemical that influences the growth and/or development of plant cells and tissues. Plant growth regulators comprise chemicals from the following five groups: auxins, cytokinins, gibberellins, abscisic acid (ABA) and ethylene. In addition to the five main groups, two other classes of chemical are often regarded as plant growth regulators: brassinosteroids and polyamines.

“Targeted mutagenesis” is mutagenesis that can be designed to alter a specific nucleotide or nucleic acid sequence, such as but not limited to, oligonucleotide-directed mutagenesis, mutagenesis using RNA-guided endonucleases (e.g. the CRISPR-technology), meganucleases, TALENs or Zinc finger technology.

The term “sequence of interest” includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said sequence of interest may be present in a double or a single strand nucleic acid molecule. The nucleic acid sequence is preferably present in a double-stranded nucleic acid molecule. The sequence of interest may be any sequence within a nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. Preferably, the sequence of interest is a small or longer contiguous stretch of nucleotides (i.e. a polynucleotide) of duplex DNA, wherein said duplex DNA further comprises a sequence complementary to the sequence of interest in the complementary strand of said duplex DNA. The sequence of interest may be, or may be part of, a gene of interest, preferably an endogenous gene of interest.

### DETAILED DESCRIPTION

The inventors discovered that by grafting a callus, e.g. derived from a protoplast, between a scion and a rootstock and subsequently inducing shoot regeneration resulted in shoots having the callus genotype. This resulted in effective regeneration of shoots from callus derived from protoplasts of recalcitrant plants, which shoots can subsequently be straightforwardly regenerated in whole plants. The present method therefore provides for an efficient protocol of regenerating whole plants from callus derived from recalcitrant plant types, and also provides for an efficient regeneration protocol for callus derived from more regenerative plant types. More in particular, the present method does not require the application of any (exogenously applied) hormones. Hence, the present method can be performed using hormone-free tissue culture media.

Therefore, in a first aspect, provided is a method of generating and selecting a shoot of a plant, wherein the method comprises the steps of:
(a) intergrafting a callus between a scion and a rootstock;
(b) allowing graft junctions to be formed between the callus and each one of the scion and the rootstock, to form a grafted union;
(c) generating a wound at or near at least one of the graft junctions;
(d) allowing the wounded grafted union to form shoots;
(e) selecting a shoot formed in step (d), wherein said shoot comprises cells derived from the callus; and optionally
(f) growing a plant from the selected shoot of step (e).

Preferably, the callus of step (a) is a protoplast-derived callus, which is understood to be a callus grown from a protoplast. Therefore, step (a) may be preceded by a step of protoplast isolation from a plant, and an optional subsequent step of callus induction. Alternatively, the callus may be a wound-derived callus generated by wounding or decapitating (i.e. removal of all preformed shoot apical meristems) a plant, and an optional subsequent step of callus induction. Said callus formation, e.g. in vitro and/or after grafting and wounding, may occur spontaneously, i.e. in the absence of one or more externally supplied plant hormones. Alternatively, said formation of callus may be induced and/or augmented in the presence of one or more plant hormones.

Preferably, the graft junctions formed in (b) are inosculated graft junctions. An inosculated graft junction is to be understood as a junction that connects the rootstock or scion with the callus in such a way that it allows nutrients and water to transfer from the rootstock to the callus tissue. Intergrafting of the callus between the rootstock and scion is to be understood herein as the process wherein the callus is placed in between a rootstock-explant and a scion-explant, thereby contacting the callus with each one of the rootstock and scion. Subsequently, graft junctions are allowed to be formed on the interface of the callus and each of the two explants. The callus will connect to both rootstock and scion by de novo formation of one or more continuous vascular strands, thus allowing e.g. nutrients, water, hormones and other metabolites to flow through the callus, effectively making the callus an integral part of the grafted plant's body.

The intergrafting in step (a) can be performed using any conventional method known to the skilled person. The callus being intergrafted between a scion and a rootstock, is to be understood herein as that the callus is brought in physical contact with at least part of the scion and part of the rootstock. Subsequently, two junctions are formed, i.e. one junction between the rootstock and the callus and one junction between the callus and the scion. The callus is preferably intergrafted along the hypocotyls or internodes of scion and rootstock, preferably under sterile conditions. Preferably, the intergrafting results in the formation of vascular tissue in the callus that connects, via the junctions, vascular tissue of the rootstock to the vascular tissue of the scion. The whole resulting structure of the rootstock, intergrafted callus and scion is called the grafted union.

The rootstock may be prepared prior to grafting by removing a shoot that comprises the apical bud, thereby rendering a “decapitated” plant, or rootstock. The removal of the shoot is preferably performed by decapitation in the hypocotyl or epicotyl, or internode. The scion may be a cotyledonary node. Preferably, said cotyledonary node is grafted on the decapitated hypocotyl rootstock with the callus placed (substantially) in between (i.e. intergrafted), forming a grafted union having two graft junctions. At each of the two graft junctions a thin strip of callus may be formed. Preferably, young plant material is used preparing the scion and/or rootstock for grafting, wherein said young plant material is preferably seedling material of between 1-4, or between 1-3 weeks after sowing, preferably using material of about 2 weeks after sowing. Preferably, the young plant material used for preparing scion and/or rootstock is seedling material having a width of between 2 and 1 mm, between 1.5 and 0.75 mm, between 1 and 0.5 mm, or between 0.5 and 0.25 mm. Preferably, seedling material is used just after development of the first true leaves. Preferably, in said grafting process, suitable steel pins, preferably sterile steel pins, are used for alignment and fixation of rootstock, callus and scion. Optionally, a steel pin is inserted in the centre of the stock- and scion with the callus fixed in between.

In addition or alternatively, ties, tapes, bands, and/or clamps may be used around the intergraft to hold the rootstock, callus and scion together, and optionally an adhesive (glue, wax or paste) may be used at edges of the graft junction for fixation. Optionally, ties, tapes, bands, and/or clamps may be used around the two grafting partners and the callus to hold them together, and optionally an adhesive (glue, wax or paste) may be used at edges of the graft junction for fixation.

Preferably, in the grafted union, the vascular tissue of the rootstock and scion are connected with each other, allowing nutrients and water to transfer from the rootstock to the scion, through the formation of vascular tissue in the grafted callus. It is to be understood that the callus and the scion and/or rootstock within the method of the invention are of plants that are naturally capable of forming graft junctions, optionally only under controlled experimental conditions. Preferably, said plants are dicotyledonous plants. Optionally, said plants are monocotyledonous plants. Preferably, the rootstock and/or scion used in the method of the invention are derived from young plant material, e.g. young plant material obtained from in vitro micropropagation, young seedling material or seeds. Method of grafting monocotyledoneous plants are provided e.g. in WO2020/099878 and WO2020/099879, which are incorporated herein by reference. For use in the method of the invention, known grafting methods are modified such that the callus is placed in between or substantially in between the scion and the rootstock.

Preferably, step (b) of graft junction formation has a duration of about 4, 5, 6, 7, 8, 9, 10, 11, 12, 13, 14, 15, 16, 17, 18, 19, or 20 days, or of about 1, 2, or 3 weeks, preferably during culturing under conditions suitable for said graft junctions to form such as exemplified herein.

Step (d) preferably comprises the formation of several shoots, i.e. more than one shoot. Therefore step (e) of the method of the invention may comprise the step of selecting a shoot from multiple shoots formed in step (d), wherein said selected shoot comprises cells of the callus. “Derived from the callus” is to be understood herein as originating from the callus by regeneration therefrom and therefore having substantially the same genotype as the cells of the callus. “Cells derived from the callus” may also be phrased as “cells of the callus”. Preferably, at least part of said selected shoot consists of cells derived from the callus. Preferably, step (e) of the method of the invention may comprise the step of selecting a shoot from multiple shoots formed in step (d), wherein at least part of said selected shoot consists of cells of the callus. Preferably, in step (d) the axis of the grafted union is perpendicular or substantially perpendicular to the earth surface and wherein the root apical meristem is closer to the earth surface as compared to the shoot apical meristem. It is known in the art, that gravity assists in endogenous hormone regulation, which is relevant for shoot formation. The position of the grafted union perpendicular or substantially perpendicular to the earth surface, wherein preferably the rootstock is placed closest to the earth surface as compared to the remaining elements of callus and (remainder of) the scion, may aid in the formation of shoots at the wound created in step (c). Preferably, the scion and the rootstock are a scion and a rootstock from seedlings, and preferably the grafted union is placed on a culture medium plate in such a way that the largest surface of the grafted union contacts the culture medium. In order for the grafted union to be substantially perpendicular to the earth surface, the culture medium plate is preferably placed in a substantially vertical position. Preferably, the culture medium is hormone free.

The callus may be of a regenerative or a recalcitrant plant. In an embodiment, the callus is from a recalcitrant plant, i.e. is a plant that fails to regenerate under normal growth conditions or that shows a poor regeneration efficiency, preferably under conditions known in the art to be optimal for regeneration. Such optimal conditions may include, but is not limited to, the presence of externally supplied growth regulators. Although within species both recalcitrant and regenerative cultivars, varieties and/or accessions may exist, typical non-limiting examples of plants known in the art to be recalcitrant are pepper (Capsicum annuum), sugarbeet (Beta vulgaris, more in particular Beta vulgaris subsp. vulgaris), soybean (Glycine max), sunflower (Helianthus annuus), cotton (Gossipium hirsutum), hemp or cannabis (Cannabis sativa), strawberry (Fragaria x ananassa), hops (Humulus lupulus), melon (Cucumis melo) and cucumber (Cucumis sativus). The calls may be of a plant which shows no or hardly any regeneration, preferably under conditions optimal for regeneration, but may also be a of plant for which regeneration efficiency can be (further) improved. Therefore, and as exemplified herein, the callus may be, but is not limited to, callus of a plant of the family Solanaceae. Likewise, the scion and/or rootstock may be, but is not limited to, of a plant that is of the family Solanaceae. However, the skilled person understands that the method of the invention can be applied to all plants, plant cells, callus or protoplasts that in some circumstance may benefit from an increase in regeneration efficiency. The callus of step (a) may be of the same or a different plant than the scion and/or rootstock of step (a). The callus of step (a) may be of a plant that is of the same or of a different species as the scion and/or rootstock of step (a). The callus of step (a) may be of a plant that is of the same or of a different genus as the scion and/or rootstock of step (a). Hence, the callus of step (a) may be derived from a plant from which also the scion and/or rootstock of step (a) are derived. Optionally, the callus of step (a) is derived from a different plant than the plant from which the scion and/or rootstock are derived, but all plants giving rise to the callus, scion and rootstock of step (a) may be from the same variety, cultivar, species, section of a genus and/or genus. Preferably, the cells of the callus of step (a) of the method of the invention have a different genotype than the cells of the scion and/or rootstock of step (a) of the method of the invention. Preferably, the callus of step (a) of the method of the invention is of a plant that is capable of exchanging genetic material through traditional breeding methods with the plant from which the scion and/or rootstock of step (a) of the method of the invention are excised. Preferably, a cell of the callus can sexually hybridize with a cell of the rootstock and/or scion.

In a preferred embodiment, the callus of step (a) of the method of the invention, or cell thereof, is of a plant that may shows less regeneration efficiency than the plant of the scion and/or rootstock of step (a) of the method of the invention, or cell or callus thereof, under conditions that are suitable, preferably optimal, for regeneration of the plant of the scion and/or rootstock, or cell or callus thereof. Such suitable and/or optimal conditions at least comprise suitable nutrient supply, optionally supplemented with hormones. Such conditions may further encompass a suitable and/or optimal temperature and/or light/dark regime. Preferably, such suitable and/or optimal conditions are applied in step (d) of the method of the invention. Preferably, the callus of step (a) shows less regeneration efficiency when exposed to similar conditions as applied in step (d) of the method of the invention, with the exception that said callus is not in contact with the rootstock and scion of step (a). These conditions are preferably conditions suitable for the plant of the scion and/or rootstock, or cell or callus thereof, to regenerate. These conditions are preferably conditions suitable for a wounded or decapitated plant of the scion and/or rootstock to induce shoots. The skilled person is aware of conditions suitable for regenerative plant cells to regenerate. Such conditions may be conditions under which the callus of step (a) normally (i.e. when not in contact with a scion and a rootstock of step (a)) does not show, are hardly shows, regeneration. Optionally, the callus of step (a) is of a plant that is of a species that shows less regeneration efficiency as the plant of the rootstock and/or scion of step (a) of the method of the invention. Alternatively, the callus of step (a) is of the same plant species as the scion and/or rootstock, but the scion and/or rootstock may be transformed and/or mutated to show increased regeneration efficiency as compared to the callus. For instance, the scion and/or rootstock may comprise a construct and/or a transgene that increase regeneration efficiency, such as, but not limited to, constructs and/or transgenes described in WO2019/211296 and WO2019/193143, which are incorporated herein by reference.

Alternatively, the callus of step (a) of the method of the invention, or cell thereof, is of a plant that may show increased regeneration efficiency as compared to the plant of the scion and/or rootstock of step (a) of the method of the invention, or cell or callus thereof, under conditions that are suitable, preferably optimal, for regeneration of the plant of the scion and/or rootstock, or cell or callus thereof. Such suitable and/or optimal conditions are preferably as defined herein. Optionally, the callus of step (a) is of a plant that is of a species that shows increased regeneration efficiency as compared to the plant of the rootstock and/or scion of step (a) of the method of the invention. Alternatively, the scion and/or rootstock are of the same plant species as the callus, but may be from a plant transformed and/or mutated to show decreased regeneration capacity as the callus. For instance, the scion and/or rootstock may be from a plant that is a non-regenerative mutant in the goblet gene (Berger Y. et al. (2009) The NAC-domain transcription factor GOBLET specifies leaflet boundaries in compound tomato leaves. Development 136 (5): 823-832), preferably said plant is a homozygous gob null mutant.

Preferably, step (d) of the method of the invention is performed under conditions suitable for the plant of the scion and/or rootstock to regenerate. Optionally, step (d) of the method of the invention may be performed under conditions known to the skilled person suitable for shoot induction of a wounded plant of the scion and/or rootstock or wounded scion and/or rootstock. Such conditions may be conditions under which the callus of step (a) normally (i.e. when not in contact or intergrafted with the scion and/or rootstock of step (a)) does not show, or hardly shows, regeneration.

Preferably, the callus of step (a) is a protoplast-derived callus and the protoplast may be of a somatic cell. Optionally, said protoplast and cells of the callus derived therefrom comprise a positive selection marker.

Preferably, the plant of the scion and/or rootstock (or cell thereof) of step (a) of the method of the invention is capable of regeneration under normal growth conditions, preferably in the absence of externally supplied (e.g. the addition of chemicals through human interference) growth regulators such as auxins and/or cytokinines. In a preferred embodiment, said conditions are at least the minimal required conditions for regeneration of the plant of the scion and/or rootstock. Optionally, said conditions are at least the suitable conditions and optionally the optimal conditions for regeneration of plant or plant cell of the scion and/or rootstock. Preferably under such conditions, the plant of the scion and/or rootstock may form de novo shoots on a multicellular tissue. The regeneration is preferably at least one of organogenesis and somatic embryogenesis. Preferably, the (regenerative) plant of the scion and/or rootstock is capable of regenerating shoots after wounding or decapitation.

The plant of the scion and/or rootstock may be a naturally occurring regenerative plant, i.e. a plant that has a natural ability to regenerate. Alternatively, the scion and/or rootstock may be genetically modified to increase the regeneration potential. Examples of genes, transgenes or constructs capable to increase the regeneration potential or capacity of plants include, but are not limited to, the genes, transgenes and constructs disclosed in WO2019/211296 and WO2019/193143, which are incorporated herein by reference. As a non-limiting example, the scion and/or rootstock may be modified to have induced or increased expression of a histidine kinase selected from the group consisting of CHK4, CHK2 and CHK3, preferably as described in WO2019/193143. In addition or alternatively, the scion and/or rootstock plant may be modified to have, preferably transiently, induced or increased expression of transcription factors associated with regeneration, preferably at least one of a WUSCHEL related homeobox protein (preferably WOX5, optionally AtWox5 of SEQ ID NO: 1), a PLETHORA protein (preferably PLT1, optionally AtPLT1 of SEQ ID NO: 2) and WOUND INDUCED DEDIFFERENTIATION 1 protein (WIND1, optionally AtWIND1 of SEQ ID NO: 3), preferably both WOX5 and PLT1, even more preferably WOX5, PLT1 and WIND1, as described in WO2019/211296. Preferably, said transcription factors are under the control of an inducible promoter and regeneration is induced by exposing the cells of the scion and/or rootstock to the agent resulting in the induction of said inducible promoter. Optionally, the scion and/or rootstock are transfected by the SHOOT REGENERATION-2 vector or the SHOOT REGENERATION vector as described in WO2019/211296. Said vector may be introduced by transient or stable transfection and regeneration may be induced by exposing the scion and/or rootstock to at least one of dexamethasone and estradiol, preferably to both dexamethasone and estradiol, as the indicated transcription factors associated with regeneration are under the control of promoters that are inducible through administration of these compounds (referred in this respect is to WO2019/211296). In addition or alternatively, the the scion and/or rootstock may have a mutation in an endogenous gene resulting in increased regeneration capacity and/or efficiency. Non-limiting examples are known in the art, e.g. the ATHB15 mutant described in Duclerq et al. (Plant biology, 2011, 13, p 317-324), the KCS1 mutant as described in Shang et al. (PNAS 2016, 113, 5101-5106), ARR mutants as described in Buechel et al. (European Journal of Cell Biology 2010, 89:279-284) and ATRXR2 mutant as described in Lee et al. (2021) Cell Reports 37, 1-13.

Optionally, the cells of the scion and/or rootstock are modified to comprise a negative selection marker.

The method as provided herein may comprise a step (g) of obtaining seed and/or progeny of the plant grown in step (f), preferably by sexual propagation and/or vegetative propagation. The sexual propagation is preferably at least one of selfing and backcrossing

Preferably, in step (e) of the method of the invention, the selected shoot that comprises cells of the callus comprises a germline progenitor cell derived from the callus. Preferably, the method further comprises step (f) and a step (g) of obtaining seed and/or plant progeny of the plant grown in step (f) by sexual propagation, optionally by selfing and/or backcrossing. Within said embodiment, the method of the invention may also be phrased as a method of generating and selecting a shoot of a plant, wherein the selected shoot comprises a germline progenitor cell of the callus, i.e. a germline progenitor cell regenerated from the callus of step (a) of the method of the invention. Germline progenitor cells are understood herein as those cells, or their clonal descendants, that will ultimately differentiate into gametes. The genotype of the germline progenitor cell therefore determines the genotype of the gamete and any genomic modification made in a germline progenitor cell will be carried on to the subsequent generation(s). Hence a transgene or mutation introduced in a germline progenitor cell is heritable, i.e. an heritable transgene or a heritable mutation. The L2-shoot meristem layer may determine the genotype of the gametes (see e.g. Filippis et al. Using a periclinal chimera to unravel layer-specific gene expression in plants, The Plant Journal, 2013, 75:1039-1049). Preferably, in step (e) the selected shoot that comprises cells of the callus comprises a germline progenitor cell of the callus. Preferably the shoot selected in step (e) comprises tissue derived from the callus of the (a), wherein preferably said tissue is (at least part of) the L2-shoot meristem layer.

The shoot selected in step (e) of the method of the invention may further comprise at least one of an L1 and an L3-shoot meristem layer derived from the callus of step (a). Optionally, said shoot comprises the L1, L2 and L3-shoot meristem layer of the callus of step (a). Alternatively, the shoot selected in step (e) of the method of the invention may comprise an L2-shoot meristem layer derived or regenerated from the callus of step (a) and at least one of the L1- and L3-shoot meristem layer derived or regenerated from the scion and/or rootstock of step (a). A meristem layer derived from the scion and/or rootstock may be regenerated from the scion and/or rootstock of step (a) via the formation of callus.

In addition or alternatively, in step (e) of the method of the invention, the selected shoot comprises cells of the callus, and wherein optionally the method further comprises step (f) and a step (g) of obtaining progeny of the plant grown in step (f) by vegetative propagation. Hence, the invention also pertains to a method of generating and selecting a shoot of a plant, wherein the shoot comprises cells giving rise to clonally propagated tissue and/or a plant part of callus of step (a), i.e. clonally propagated tissue and/or plant part regenerated from the callus of step (a) of the method of the invention.

Therefore, preferably the method of the invention comprises a step (e) of selecting a shoot, wherein at least part of said shoot consists of cells of the callus (i.e. being regenerated from the callus of step (a)), and wherein preferably said part is at least one of:
i) a tissue comprising germline progenitor cells; and
ii) a tissue comprising cells giving rise to a clonally (vegetatively) propagated tissue and/or a clonally (vegetatively) propagated plant part.

Clonally propagated tissue and/or plant part is understood herein as a tissue and/or plant part that can be used for clonal propagating into offspring, i.e. a plant of a subsequent generation. Such tissue and/or plant part may be, but is not limited to, a tuber, bulb, corm, cormel, sucker, slip, crown, bulbil, rhizome, apical portion of stem, shoot or root cutting, basal knob or truncheon, stolon, tuberous stem cutting or eye, (clonally propagated) seed, and the like. The genotype of the (cells giving rise to) clonally propagated plant part or tissue therefore determines the genotype of offspring of clonally propagated plants and any genomic modification made in (cells giving rise to) this tissue or part may be carried on to the subsequent generation(s). Hence a transgene or mutation made in (cells giving rise to) a clonally propagated plant part is a heritable transgene or mutation.

The shoots, or at least one shoot, grown in step (d) and selected in step (e) of the method of the invention may be adventitious shoots, or at least one adventitious shoot.

The callus in step (a) is contacted to a scion and a rootstock to form an grafted union. Optionally, the scion and the rootstock are from the same plant or from different plants. Alternatively, the scion and the rootstock maybe from similar plants, wherein similar plants are to be understood as plants of the same species, variety or cross, even more preferably plants that have substantially the same genotype. Preferably, the plants providing the scion and the rootstock have been obtained by vegetative propagation of the same plant and hence are genetically identical. Optionally scion and rootstock are from two plants that are of the same variation, cultivars, species, section of a genus or genus. In case the scion and the rootstock are from the same or similar plant, in step (a) of the method of the invention the callus of a first plant may be contacted with scion and rootstock of a second plant, wherein optionally the first and second plant are of a different cross, variety or species. Preferably, said first and second plant are of the same section of genus or genus. Alternatively, the callus may be derived from the same or similar plant as the scion and the rootstock. For instance, the callus of step (a) may be produced from a protoplast isolated from a leaf of a plant, wherein said plant (or a plant of the same variety, species or cross) is subsequently used for the excision of a rootstock and scion for integrafting the callus in step (a). Alternatively, the callus of step (a) may be produced from a protoplast isolated from a leaf of a first plant, and the scion and the rootstock are excised from a second plant, wherein said second plant is of the same cross, species or variety plant as the first plant.

In case the scion is from a plant that is a different plant as the rootstock, in step (a) of the method of the invention the callus of a first plant may be contacted with scion of a second plant and rootstock of a third plant. Alternatively, the callus may be derived from the same plant as the scion or the rootstock. Hence, in step (a) of the method of the invention the callus of a first plant may contacted with scion of the first plant and rootstock of a second said plant, or in step (a) of the method of the invention the callus of a first plant may be contacted with rootstock of the first plant and scion of a second said plant. Preferably, the first, second and optionally third plant are of the same section of a genus or genus, preferably of the same species, variety or cross, even more preferably, have substantially the same genotype.

In step (c), a wound is generated at or near at least one of the graft junctions. This is performed after formation of the graft junctions in step (b). The wounding may be performed by cutting, which induces the production of callus, and adventitious shoots. Among these adventitious shoots, shoots comprising or consisting of cells of, or derived of, the callus of step (a) can appear spontaneously. The cells of, or derived of the callus of step (a), may comprise a germline progenitor cell derived and/or clonally propagated plant tissue and/or plant parts.

The wound is preferably made at the intersection between the callus and the rootstock or scion of the grafted union, i.e. at or near the graft junction formed between the callus and at least one of the rootstock or scion. Preferably, the wound is made at or near the graft junction formed between the callus and the scion. The wound is preferably such that substantially all of the scion is removed. More in particular, preferably a cut is made just above the junction between the callus and the scion rendering a thin layer of scion cells at the scion side of the callus-scion-junction. Alternatively or in addition, the wound may be at or near the graft junction formed between the rootstock and the callus, preferably just below the junction between the callus and the rootstock rendering a thin layer of rootstock cells at the rootstock side of the callus-rootstock-junction. Hence, preferably step (c) may comprise a step of generating a wound at or near at least one of the graft junctions and allowing callus to be formed at the wounded graft junction.

The wound may be a complete cut, e.g. a transverse cut, separating the graft into two plant parts. Optionally, the wound (cut) does not completely separate the graft union into two plant parts, but is sufficient to initiate and/or stimulate the production of callus. Preferably, a shoot is grown from said callus, wherein said shoot may comprise or consist of tissue regenerated from the callus of step (a). Optionally, at least part of said shoot consist of cells regenerated from the callus. This particular method is preferably practiced under ambient conditions, in a growth room or greenhouse.

Therefore, optionally, the contacting of step (a) is performed by grafting a callus on the rootstock, and optionally further grafting a scion on said callus, and allowing the, optionally two, graft junction(s) to formed in step (b). Said method further comprises the step (c) of generating a wound just at or near the graft junction, preferably the graft junction between the scion and/or rootstock and the callus, allowing or inducing (further) callus to be formed at the (wounded) graft junction and allowing a shoot to grow from said callus, and wherein said shoot comprises cells regenerated of the callus of step (a). Optionally, at least part of said shoot consist of cells regenerated of the callus of step (a).

Preferably, in the method of the invention, at least one (adventitious) shoot comprising or consisting of tissue regenerated from the callus is allowed to form, preferably said tissue comprises a germline progenitor cell, and/or gives rise to a clonally propagated tissue and/or a clonally propagated plant part.

Step (d) is performed under conditions suitable for shoot formation, optionally using conditions known by the skilled person that are suitable for shoot regeneration. Preferably, these conditions are at least the minimal requirements for shoot regeneration of a (wounded) plant from which the scion and/or rootstock are derived, which in general at least include normal growth conditions of said plant. Preferably, step (d) comprises the formation of callus prior to shoot formation. Therefore, step (d) may comprise the sub-steps of (d1) allowing or inducing (further) callus formation at or near the wounded graft junction; and (d2) allowing a shoot to grow from said callus, wherein optionally the culturing conditions of (d1) and (d2) are different. More in particular, step (d1) may be performed under conditions suitable for at least the plant (preferably the plant from which the scion and/or rootstock are derived) to form callus; and step (d2) may be performed under conditions suitable for callus of said plant to form shoots. The (further) callus and shoot formed in step (d1) and (d2) preferably comprise cells derived from the callus of step (a). Optionally, the (further) callus and shoot formed in step (d1) and (d2) consist of cells derived from the callus of step (a) . . . . The skilled person is aware of conditions suitable for callus and/or shoot regeneration. Preferably, in step (d) in addition to cells of the callus of step (a), also cells of the scion and/or rootstock regenerate to form callus and/or shoot. Therefore, optionally, in step (d), (d1) and/or (d2) callus and scion cells and/or rootstock cells co-regenerate.

Callus may be formed preceding the regeneration process of step (d) by (shoot) organogenesis or somatic embryogenesis. The amount of formed callus may be dependent on e.g. the plant species used in the method of the invention and/or the used conditions that allow for shoot formation. Said callus formation, e.g. in vitro and/or after grafting and wounding, may occur spontaneously, i.e. in the absence of one or more externally supplied plant hormones. Similarly, the formation of shoots in step (d), e.g. after optional callus formation, may occur spontaneously, thus in the absence of one or more externally supplied plant hormones. Alternatively, said formation of callus may be induced and/or augmented in the presence of one or more plant hormones. Alternatively or in addition, the formation of shoots in step (d) may be induced and/or augmented in the presence of one or more plant hormones.

In a particular embodiment, formation of (further) callus is minimal between the wounding step (c) and the formation of a shoot in step (d) of the method of the invention. This is in particular preferred in case callus of step (a) is of a plant that is less regenerative as the plant from which the scion and/or rootstock are excised, in order to avoid the cells of scion and/or rootstock to outcompete the cells of the callus of step (a). A minimal callus stage may therefore increase the chance of growing a shoot that comprises or consists (at least partly) of cells of the callus of step (a).

For the induction of shoot regeneration in plant tissues, a combination of one or more plant hormones, preferably cytokinins and/or one or more auxins may be employed.

The cytokinin that may be used in the method of the invention can be an adenine-type cytokinin or a phenylurea-type cytokinin. Similarly, the cytokinin can be a naturally produced phytohormone or can be a synthesized compound. The adenine-type cytokinin can be a phytohormone that is synthesized in at least one of roots, seeds and fruits. In addition, cambium and other actively dividing tissues can also synthesize cytokinins. A non-limiting example of a naturally occurring adenine-type cytokinin is Zeatin as well as its metabolic precursor 2iP. Non-limiting examples of synthetic adenine-type cytokinins are kinetin and 6-benzylaminopurine (BAP). Substituted urea compounds, such as thidiazuron and CPPU do not occur in plants but can act as cytokinins in tissue culture. The adenine-type cytokinin can be selected from the group consisting of kinetin, zeatin, trans-zeatin, cis-zeatin, dihydrozeatin, 6-benzylaminopurine and 2iP, and combinations thereof. The phenylurea-type cytokinin can be diphenylurea or thidiazuron. It is known in the art that the type of added cytokinin is dependent on the type of plant cell and the skilled person can straightforwardly select the suitable cytokinin(s), if needed.

Alternatively or in addition, the plant hormone may be an auxin. The auxin can be an endogenously synthesized auxin. The endogenously synthesized auxin can be selected from the group consisting of indole-3-acetic acid (IAA), 4-chloroindole-3-acetic acid, phenylacetic acid, indole-3-butyric acid and indole-3-propionic acid. The auxin can be a synthetic auxin, e.g. an auxin analog. The synthetic auxin can be at least one of 1-naphthaleneacetic acid, 2,4-dichlorophenoxyacetic acid (2,4-D), α-Naphthalene acetic acid (α-NAA), 2-Methoxy-3,6-dichlorobenzoic acid (dicamba), 4-Amino-3,5,6-trichloropicolinic acid (tordon or picloram), 1-naphthaleneacetic acid (NAA), indole-3-butyric acid (IBA) and 2,4,5-trichlorophenoxyacetic acid (2,4,5-T). The auxin can be 1-naphthaleneacetic acid (NAA).

For shoot organogenesis by a combination of cytokinin and auxin the cytokinin to auxin ratio preferably is >1 (Dodds, J H and Roberts, LW (1985) Experiments in plant tissue culture. Cambridge University Press, Cambridge, UK).

Optionally, in step (d) first callus formation is stimulated (step d1) and subsequently shoot formation is stimulated (step d2). Step (d1) may be performed using conditions allowing for callus formation of the plant from which the scion and/or rootstock is derived (also indicated as “the plant of the of the scion and/or rootstock”). Optionally, step (d1) is performed using minimal conditions allowing for callus formation of the plant of the scion and/or rootstock. In a preferred embodiment, step (d1) is performed using optimal conditions for callus formation of the plant of the scion and/or rootstock. Step (d2) may be performed using conditions allowing for shoot formation of the plant of the scion and/or rootstock. Optionally, step (d2) is performed using minimal conditions allowing for shoot formation of the plant of the scion and/or rootstock. In a preferred embodiment, step (d2) is performed using optimal conditions for shoot formation of the plant of the scion and/or rootstock.

The method of the invention may further comprise a step (f) of growing a plant from the shoot selected in step (e).

Optionally, in step (e) of the method of the invention, a shoot is selected that comprises germline progenitor cells derived from the callus of step (a). Such shoot can give rise to a plant comprising germline progenitor cells and/or germline cells (e.g. gametes, egg cell, sperm cell) that are derived from the callus of step (a). Germline cells may form gametes for sexual reproduction. Such plant can be subsequently used to produce seed, wherein said seed comprises an embryo, and wherein at least part of the genotype of the embryo is derived from the callus step (a) of the method of the invention, optionally the seed is obtained by selfing or backcrossing.

Optionally, in step (e) of the method of the invention, a shoot is selected that comprises cells that may give rise to clonally propagated tissue or plant parts derived from the callus of step (a) of the method of the invention. Such shoot can give rise to a plant comprising plant parts derived from the callus of step (a) that can be used for clonal propagation. Such plant part has the same or substantially the same genotype as the callus of step (a) of the method of the invention.

The selected shoot may be substantially free of cells of the scion and/or rootstock of step (a). Such shoot may consist of cells of derived from the callus of step (a) and can be used to produce a plant by (vegetative) propagation of said shoot, i.e. by growing a whole plant from said shoot. The step of selecting the shoot can be performed using any conventional method known to the skilled person.

The selection may comprise a step of determining a phenotypic characteristic and/or a molecular marker that is present in the cells of callus of step (a) and/or present in a shoot meristem layer of a plant of which said callus is derived, but absent in the cells of the scion and/or rootstock and/or absent in a shoot meristem layer of the plant from which the scion and/or rootstock are excised. Alternatively or in addition, the selection may comprise a step of determining a phenotypic characteristic and/or a molecular marker that is absent in the cells of the plant from which the callus of step (a) is derived and/or absent in a shoot meristem layer of said plant, but is present in the cells of the plant and/or present in a shoot meristem layer of the plant from which the scion and/or rootstock are excised. Preferably, the selection may comprise a step of determining a phenotypic characteristic and/or a molecular marker that is present in the germline progenitor cells and/or the clonally propagated plant part of plant from which the callus of step (a) is derived, but not present in the germline progenitor cells and/or the clonally propagated tissue and/or plant part of the second plant. Alternatively or in addition, the selection may comprise a step of determining a phenotypic characteristic and/or a molecular marker that is absent in the germline progenitor cells and/or the clonally propagated tissue and/or plant part of the plant from which the callus of step (a) is derived, but is present in the germline progenitor cells and/or the clonally propagated tissue and/or plant part of the plant from which the scion and/or rootstock are excised. The molecular marker is preferably a genomic sequence, that is present either in the plant from which the callus of step (a) is derived or in the plant from which the scion and/or rootstock are excised.

Alternatively or in addition, step (e) and/or (f) may comprise a step of bringing the (regenerated) shoot into contact with a compound that is toxic for (plant) cells that express a negative selection marker. In this embodiment, a negative selection marker may be expressed in cells of the plant from which the scion and/or rootstock are excised, preferably a negative selection marker is expressed in at least the germline progenitor cells and/or the clonally propagated tissue and/or plant part of said plant. Optionally, a toxic selection marker is encoded by (the genome of) cells of the plant from which the scion and/or rootstock are excised, optionally under the control of an inducible promoter. By exposure of such cells to a substance activating the inducible promoter, the toxic selection marker is expressed and preferably these cells die. Optionally, a precursor of a toxic selection marker is encoded by the (genome of) in the cells of the scion and/or rootstock. By exposure of such cells to a substance that activates the conversion of the precursor to a toxic component, preferably thereby killing these cells.

Alternatively or in addition, step (e) and/or (f) may comprise a step of bringing the (regenerated) shoot into contact with a compound that is toxic for (plant) cells, but can be converted into a non-toxic compound by the expression of a positive selection marker. In this embodiment, a positive selection marker may be expressed in a shoot meristem layer of the plant from which the callus of step (a) is derived, preferably a positive selection marker is expressed in at least the germline progenitor cells and/or the clonally propagated tissue and/or plant part of the said plant.

In a preferred embodiment, at least one or more germline progenitor cells and/or the clonally propagated tissue and/or plant parts of the generated shoot selected in step (e) of the method of the invention comprise a transgene or a mutation in a sequence of interest. Preferably, at least one of the L1-, L2- and/or L3-shoot meristem layer of the generated shoot comprises a transgene or mutation in a sequence of interest. Preferably, at least the L2-shoot meristem layer of the generated shoot comprises a transgene or mutation in a sequence of interest.

The transgene or mutation may be present in a cell of, or derived from, the callus of step (a) of the method of the invention and optionally in a cell of, or derived from, the scion and/or rootstock of the second plant of step (a) of the method of the invention. Preferably, the transgene or mutation is present in all or substantially all cells of the callus of step (a) of the method of the invention. Preferably, the transgene or mutation is present at least in a germline progenitor cell and/or the clonally propagated tissue and/or plant part of the generated shoot selected in step (e) of the method of the invention. Optionally, the transgene or mutation is present in all or substantially all cells of the generated shoot selected in step (e), wherein said cells are cells regenerated from the callus of step (a) of the method of the invention. Preferably, at least the L2-shoot meristem layer of the generated shoot comprises a transgene or mutation in a sequence of interest and wherein at least the L2-shoot meristem layer is is regenerated from the callus of step (a). Subsequent seed produced from such shoot may comprise said transgene or mutation, preferably within the embryo of said seed.

Therefore preferably the method of the invention comprises the step of introducing transgene or a mutation in a sequence of interest in one or more cells of the callus of step (a). Alternatively or in addition, the method of the invention (further) comprises the step of introducing a transgene or mutation in a sequence of interest in one or more cells originating from the callus of step (a) and present in the shoot formed in step (d), optionally in the callus formed in step (d1).

The transgene or mutation may be introduced into the one or more cells of the callus of step (a) before the callus is contacted (intergrafted) with the scion and the rootstock of step (a). Optionally, the transgene or mutation may be introduced into a protoplast that is subsequently developed into the callus for used in step (a). Alternatively, the transgene or mutation can be introduced in one or at least part of the callus of step (a) and/or wounded plant tissue from which the callus for use in step (a) is subsequently formed. Similarly, the transgene or mutation can be introduced in one or more cells of a plant and cells said plant carrying the transgene or mutation may be induced to form callus for use in step (a).

Alternatively or in addition, the transgene or mutation may be introduced into cells of the callus of step (a) after contacting the callus with at least one of the scion or rootstock of step (a). The transgene or mutation is preferably introduced before shoot formation. Thus preferably, the step of introducing a mutation is prior to step (d), or (d2) of the method as defined herein, but may be during or after step (c) or (d1). Optionally, the transgene or mutation may also be introduced into one or more cells of the at least one of the scion or rootstock of step (a).

Optionally the graft junction(s) are first healed, prior to introducing a transgene or mutation in at least one or more cells of, or derived of, the callus of step (a). Optionally, the graft union is first cut or “wounded” prior to introducing a transgene or mutation in at least one or more cells of, or derived of, the callus of step (a). Optionally, (further) callus formation is first induced by the wounding, prior to introducing a transgene or mutation in at least one or more cells of, or derived of, the callus of step (a).

Preferably, the shoot selected in step (e) of the method of the invention comprises a transgene or mutation in the sequence of interest in a cell regenerated from the callus of of step (a) of the method of the invention, i.e. apart from the transgene or mutation, having the genotype of said callus. The shoot preferably comprises the transgene or mutation in at least one of the L1-, L2- and L3-shoot meristem layer regenerated from the callus of step (a). In other words, at least one of the L1-, L2- and L3-shoot meristem layer of the shoot selected in (e) has, apart from the transgene or mutation, the genotype of the callus of step (a). Preferably, at least the germline progenitor cells and/or the clonally propagated tissue and/or plant part of the selected shoot are regenerated from the callus of step (a) of the method of the invention (i.e. have the genotype of the callus of step (a)) and comprise a transgene or mutation in a gene of interest. Hence, a preferred method of the invention is a method of generating and selecting a shoot of a plant, wherein the shoot comprises germline progenitor cells and/or comprises cells giving rise to a clonally propagated tissue and/or plant part derived from the callus of step (a) and wherein the one or more of the germline progenitor cells and/or clonally propagated plant tissue and/or plant parts comprise a transgene or mutation in a sequence of interest. Preferably, all or substantially all germline progenitor cells and/or clonally propagated tissues and/or plant parts comprise the transgene or mutation in the sequence of interest.

The transgene or mutation may be present in at least the L2-shoot meristem layer. Hence, a preferred method of the invention is a method of generating and selecting a shoot of a plant, wherein the shoot comprises an L2-shoot meristem layer derived from the callus of step (a) and wherein the one or more cells of the L2-shoot meristem layer comprises a transgene or mutation in a sequence of interest. Preferably, all or substantially all cells of at least the L2-shoot meristem layer comprises the transgene or mutation in a sequence of interest. Optionally, the transgene or mutation is present in all or substantially all cells of the generated shoot selected in step (c), wherein said cells are cells regenerated from the callus of step (a) of the method of the invention. Optionally, shoot selected in step (e) have substantially the same genotype as the cells of the callus of step (a), preferably comprising the transgene or mutation.

An introduction of a transgene or a mutation in a sequence of interest in the method of the invention preferably results in a one or more improved phenotypic properties, such as but not limited to an increased yield, disease resistance, agronomic traits, abiotic traits, protein composition, oil composition, starch composition, insect resistance, fertility, silage, and morphological traits.

The transgene may be introduced by stable or transgenic transfection using any method known by the person skilled in the art to transfect a plant, plant part, callus, plant cell or protoplast.

A mutation is to be understood herein as an alteration in the genome, preferably in the genetic code, either in nucleotide sequence (insertion, deletion or substitution of one or more nucleotides, or a chromosomal translocation) or epigenetic alterations such as a change in methylation. A mutation may be introduced by random mutagenesis or targeted mutagenesis, the latter also being referred to as programmed genome editing. Random mutagenesis may be, but is not limited to, chemical mutagenesis and gamma radiation. Non-limiting examples of chemical mutagenesis include, but are not limited to, EMS (ethyl methanesulfonate), MMS (methyl methanesulfonate), NaN3 (sodium azide) D), ENU (N-ethyl-N-nitrosourea), AzaC (azacytidine) and NQO (4-nitroquinoline 1-oxide). Optionally, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate a mutation in a cell of a recalcitrant plant. TILLING uses traditional chemical mutagenesis (e.g. EMS mutagenesis) followed by high-throughput screening for mutations. Thus, plants, seeds and tissues comprising a gene having one or more of the desired mutations may be obtained using TILLING. Preferably, plants, seeds and tissues comprising a gene having one or more of the desired mutations may be obtained using KeyPoint® Breeding as described in WO2007/037678, which is incorporated herein by reference.

Targeted mutagenesis or programmed genome editing is mutagenesis that can be designed to alter a specific nucleotides or nucleic acid sequence, such as but not limited to, oligo-directed mutagenesis, RNA-guided endonucleases (e.g. the CRISPR-technology), TALENS, meganucleases or Zinc finger technology.

Preferably, the targeted mutagenesis is introduced by a site-specific protein, preferably a site-specific endonuclease. The site-specific endonuclease is preferably at least one of a CRISPR-protein complexed with a guide RNA, a TALEN, a Zinc Finger Protein, a meganuclease and an Argonaute complex. Preferably, the site-specific endonuclease is a CRISPR protein complexed with a guide RNA.

The CRISPR-protein that is part of the CRISPR protein complex for use in the method of the invention is preferably at least one of a CRISPR-endonuclease, CRISPR-nickase and a CRISPR-deaminase. Preferably, the CRISPR-protein is a CRISPR-endonuclease.

The CRISPR-protein can be any suitable CRISPR-protein known in the art. Optionally, the CRISPR-protein comprises a nuclear localisation signal (NLS) to direct the CRISPR-protein to the nucleus of the plant cell. Any known nuclear localisation signal would be suitable for use in the invention. Preferred nuclear localisation signals include, but are not limited to the NLS of the SV40 Large T-antigen MEDPTMAPKKKRKV (SEQ ID NO: 4) and the NLS of nucleoplasmin KRPAATKKAGQAKKKK (SEQ ID NO: 5).

A CRISPR-endonuclease comprises a nuclease domain and at least one domain that interacts with a guide RNA. When complexed with a guide RNA, the CRISPR protein complex is directed to a specific nucleic acid sequence by a guide RNA. The guide RNA interacts with the CRISPR-endonuclease as well as with a target-specific nucleic acid sequence, such that, once directed to the site comprising the target nucleic acid sequence via the guide sequence, the CRISPR-endonuclease is able to introduce a double-stranded break at the target site.

In case the CRISPR-protein is a CRISPR-endonuclease, both domains of the nuclease are catalytically active and the protein is able to introduce a double-stranded break at the target site. In case the CRISPR-protein is a CRISPR-nickase, one domain of the nuclease is catalytically active and one domain is catalytically inactive, and the protein is able to introduce a single-stranded break at the target site.

The skilled person is well aware of how to design a guide RNA in a manner that it, when combined with a CRISPR-endonuclease or CRISPR-nickase, effects the introduction of a single- or double-stranded break at a predefined site in the nucleic acid molecule.

CRISPR-proteins can generally be categorized into six major types (Type I-VI), which are further subdivided into subtypes, based on core element content and sequences (Makarova et al, 2011, Nat Rev Microbiol 9:467-77 and Wright et al, 2016, Cell 164 (1-2): 29-44). In general, the two key elements of a CRISPR-protein complex is a CRISPR-protein and a guide RNA.

Type II CRISPR-protein complexes include a signature Cas9 protein, a single protein (about 160 KDa), capable of specifically cleaving duplex DNA. The Cas9 protein typically contains two nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096): 816-821). The Cas9 protein is an example of a CAS protein of the type II CRISPR-CAS protein complex and forms an endonuclease, when combined with the crRNA and a second RNA termed the trans-activating crRNA (tracrRNA). The crRNA and tracrRNA function together as the guide RNA. The CRISPR-protein complex introduces DNA double strand breaks (DSBs) at the position in the genome defined by the crRNA. Jinek et al. (2012, Science 337:816-820) demonstrated that a single chain chimeric guide RNA (herein defined as a “sgRNA” or “single guide RNA”) produced by fusing an essential portion of the crRNA and tracrRNA was able to form a functional CRISPR-protein complex in combination with the Cas9 protein.

A Type V CRISPR-protein complex has been described, the Clustered Regularly Interspaced Short Palindromic Repeats from Prevotella and Francisella 1 or CRISPR/Cpf1. Cpf1 genes are associated with the CRISPR locus and coding for an endonuclease that use a crRNA to target DNA. Cpf1 is a smaller endonuclease than Cas9, which may overcome some of the CRISPR-Cas9 system limitations. Cpf1 is a single RNA-guided endonuclease lacking tracrRNA, and it utilizes a T-rich protospacer-adjacent motif. Cpf1 cleaves DNA via a staggered DNA double-stranded break (Zetsche et al (2015) Cell 163 (3): 759-771). The type V CRISPR-Protein system preferably includes at least one of Cpf1, C2c1 and C2c3.

The CRISPR-protein complex for use in the invention may comprise any CRISPR-protein as defined herein above. Preferably, the CRISPR-protein is a Type II CRISPR-protein, preferably a Type II CRISPR-endonuclease, e.g., Cas9 (e.g., the protein of SEQ ID NO: 6, encoded by SEQ ID NO: 7, or the protein of SEQ ID NO: 8) or a Type V CRISPR-protein, preferably a Type V CRISPR-endonuclease, e.g. Cpf1 (e.g., the protein of SEQ ID NO: 9, encoded by SEQ ID NO: 10) or Mad7 (e.g. the protein of SEQ ID NO: 11 or 12), or a protein derived thereof, having preferably at least about 70%, 80%, 90%, 91%, 92%, 93%, 94%, 95%, 96%, 97%, 98%, or 99% sequence identity to said protein over its whole length.

Preferably, the CRISPR-protein is a Type II CRISPR-endonuclease, preferably a Cas9 endonuclease.

The skilled person knows how to find and prepare a CRISPR-protein for use in the method of the invention. In the prior art, numerous reports are available on its design and use. See for example the review by Haeussler et al (J Genet Genomics. (2016)43(5):239-50. doi: 10.1016/j.jgg.2016.04.008.) on the design of guide RNA and its combined use with a CAS-protein (originally obtained from S. pyogenes), or the review by Lee et al. (Plant Biotechnology Journal (2016) 14(2) 448-462).

In general, a CRISPR-endonuclease, such as Cas9, comprises two catalytically active nuclease domains. For example, a Cas9 protein can comprise a RuvC-like nuclease domain and an HNH-like nuclease domain. The RuvC and HNH domains work together, both cutting a single strand, to make a double-stranded break in DNA. (Jinek et al., Science, 337:816-821).

A dead CRISPR-endonuclease comprises modifications such that none of the nuclease domains shows cleavage activity. The CRISPR-nickase may be a variant of the CRISPR-endonuclease wherein one of the nuclease domains is mutated such that it is no longer functional (i.e., the nuclease activity is absent). An example is a SpCas9 variant having either the D10A or H840A mutation.

The CRISPR-protein may comprise or consist of a whole type II or type V CRISPR-protein or a variant or functional fragment thereof. Preferably such fragment binds the guide RNA and maintains, at least partly, endonuclease activity.

Preferably, the CRISPR-protein for use in the method of the invention is a Cas9 protein. The Cas9 protein may be derived from the bacteria Streptococcus pyogenes (SpCas9; NCBI Reference Sequence NC_017053.1; UniProtKB-Q99ZW2), Geobacillus thermodenitrificans (UniProtKB-A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis/(NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9, e.g. the SpCas9_D10A or SpCas9_H840A, or a Cas9 having equivalent substitutions at positions corresponding to D10 or H840 in the SpCas9 protein, rendering a nickase.

The CRISPR-protein for use in the method of the invention may be, or may be derived from, Cpf1, e.g. Cpf1 from Acidaminococcus sp; UniProtKB-U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165(4): 949-962). In this variant, there is an arginine to alanine (R1226A) conversion in the NUC-domain, which inactivates the NUC-domain.

The CRISPR-protein for use in the method of the invention may be, or may be derived from, CRISPR-CasΦ, a nuclease that is about half the size of Cas9. CRISPR-CasΦ uses a single crRNA for targeting and cleaving the nucleic acid as is described e.g. in Pausch et al (CRISPR-CasΦ from huge phages is a hypercompact genome editor, Science (2020); 369 (6501): 333-337).

An active, partly inactive or dead CRISPR-protein may be used in the method of the invention, e.g. to guide a fused functional domain as detailed herein to a specific site in the DNA as determined by the guide RNA.

Hence, the CRISPR-protein may be fused to a functional domain. Optionally, such functional domain is for epigenetic modification, for example a histone modification domain. The domains for epigenetic modification can be selected from the group consisting of a methyltransferase, a demethylase, a deacetylase, a methylase, a deacetylase, a deoxygenase, a glycosylase and an acetylase (Cano-Rodriguez et al, Curr Genet Med Rep (2016) 4:170-179). The methyltransferase may be selected from the group consisting of G9a, Suv39 h1, DNMT3, PRDM9 and Dot1L. The demethylase may be LSD1. The deacetylase may be SIRT6 or SIRT3. The methylase may be at least one of KYP, TgSET8 and NUE. The deacetylase may be selected from the group consisting of HDAC8, RPD3, Sir2a and Sin3a. The deoxygenase may be at least one of TET1, TET2 and TET3, preferably TET1cd (Gallego-Bartolome J et al, Proc Natl Acad Sci USA. (2018); 115(9):E2125-E2134). The glycosylase may be TDG. The acetylase may be p300.

Optionally, the functional domain is a deaminase, or functional fragment thereof, selected from the group consisting of an apolipoprotein B mRNA-editing complex (APOBEC) family deaminase, an activation-induced cytosine deaminase (AID), an ACF1/ASE deaminase, an adenine deaminase, and an ADAT family deaminase. Alternatively or in addition, the deaminase or functional fragment thereof may be ADAR1 or ADAR2, or a variant thereof.

The apolipoprotein B mRNA-editing complex (APOBEC) family of cytosine deaminase enzymes encompasses eleven proteins that serve to initiate mutagenesis in a controlled and beneficial manner. Preferably, the APOBEC deaminase is selected from the group consisting of APOBEC1, APOBEC2, APOBEC3A, APOBEC3B, APOBEC3C, APOBEC3D, APOBEC3F, APOBEC3G, APOBEC3H, APOBEC4 and Activation-induced (cytidine) deaminase. Preferably, the cytosine deaminase of the APOBEC family is activation-induced cytosine (or cytidine) deaminase (AID) or apolipoprotein B editing complex 3 (APOBEC3). Preferably, the deaminase domain fused to the CRISPR-protein an APOBEC1 family deaminase.

Another exemplary suitable type of deaminase domain that may be fused to the CRISPR-system nuclease is an adenine or adenosine deaminase, for example an ADAT family of adenine deaminase. Further, the adenine deaminase may be TadA or a variant thereof, preferably as described in Gaudelli et al., 2017 (Gaudelli et al. 2017 Nature 551:464-471). Further, the CRISPR-system nuclease may be fused to an adenine deaminase domain, e.g. derived from ADAR1 or ADAR2. The deaminase domain of the present invention may comprise or consist of a whole deaminase protein or a fragment thereof which has catalytic activity. Preferably, the deaminase domain has deaminase activity. Optionally, the CRISPR-protein is further fused to an UDG inhibitor (UGI) domain.

The CRISPR-protein for use in the method of the invention is complexed with a guide RNA molecule, which guides the CRISPR-protein to a specific location in the genome of a plant cell to achieve a targeted genomic modification. Preferably the plant cell is a cell of a recalcitrant plant. Optionally, the plant cell is a germline or germline progenitor cell and/or a cell giving rise to a clonally propagated tissue and/or plant part of a recalcitrant plant.

The complex comprising a CRISPR-protein and a guide RNA may also be annotated as a ribonucleoprotein complex.

The guide RNA molecule directs the complex to a defined target site in a double-stranded nucleic acid molecule, also named the protospacer sequence. The guide RNA molecule comprises a sequence for targeting the CRISPR-protein complex to a protospacer sequence that is preferably near, at or within a sequence of interest in the genome of the plant cell. The guide RNA can be a single guide (sg)RNA or the combination of a crRNA and a tracrRNA (e.g. for Cas9) or a crRNA only (e.g. in case of Cpf1 and CasΦ).

The CRISPR-protein complex for use in the method of the invention may thus comprise a guide RNA molecule, wherein the guide RNA molecule comprises a combination of a crRNA and a tracrRNA, and wherein preferably the CRISPR-protein is Cas9. The crRNA and tracrRNA are preferably combined into a sgRNA (single guide RNA). Alternatively, the CRISPR-protein complex for use in the method of the invention may comprise a guide RNA molecule, wherein the guide RNA molecule comprises a crRNA, and wherein preferably the CRISPR protein is Cpf1 or CasΦ.

The guide RNA molecule for use in a method of the invention may comprise a sequence that can hybridize to or near a sequence of interest, preferably a sequence of interest as defined herein. The guide RNA molecule may comprise a nucleotide sequence that is fully complementary to a sequence in the sequence of interest, i.e. the sequence of interest comprises a protospacer sequence. Alternatively or in addition, the guide RNA molecule for use in the method of the invention may comprise a sequence that can hybridize to or near the complement of a sequence of interest.

The part of the crRNA that is complementary to the protospacer sequence is designed to have sufficient complementarity with the protospacer sequence to hybridize with the protospacer sequence and direct sequence-specific binding of a complexed CRISPR protein. The protospacer sequence is preferably adjacent to a protospacer adjacent motif (PAM) sequence, which PAM sequence may interact with the CRISPR protein of the RNA-guided CRISPR-protein complex. For instance, in case the CRISPR protein is S. pyogenes Cas9, the PAM sequence preferably is 5′-NGG-3′, wherein N can be any one of T, G, A or C.

The skilled person is capable of engineering the crRNA to target any desired sequence, preferably by engineering the sequence to be at least partly complementary to any desired protospacer sequence, in order to hybridize thereto. Preferably, the complementarity between part of a crRNA sequence and its corresponding protospacer sequence, when optimally aligned using a suitable alignment algorithm, is at least 70%, 80%, 85%, 90%, 91%, 92%, 93%, 94%, 95%, 96%, 97%, 98% or 100%. The part of the crRNA sequence that is complementary to the protospacer sequence may be at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some preferred embodiments, the sequence complementary to the sequence of interest is less than about 75, 50, 45, 40, 35, 30, 25, 20 nucleotides in length. Preferably, the length of the sequence complementary to the sequence of interest is at least 17 nucleotides. Preferably the complementary crRNA sequence is about 10-30 nucleotides in length, about 17-25 nucleotides in length or about 15-21 nucleotides in length. Preferably the part of the crRNA that is complementary to the protospacer sequence is 15, 16, 17, 18, 19, 20, 21, 22, 23, 24 or 25 nucleotides in length, preferably 20 or 21 nucleotides, preferably 20 nucleotides.

Molecules suitable as crRNA and tracrRNA are well known in the art (see e.g., WO2013142578 and Jinek et al., Science (2012) 337, 816-821). The crRNA and tracrRNA in the guide RNA molecule can be linked to together to form a single guide (sg)RNA. The crRNA and tracrRNA can be linked, preferably covalently linked, using any conventional method known in the art. Covalent linkage of the crRNA and tracrRNA is e.g. described in Jinek et al. (supra) and WO13/176772, which are incorporated herein by reference. The crRNA and tracrRNA can be covalently linked using e.g. linker nucleotides or via direct covalent linkage of the 3′ end of the crRNA and the 5′ end of the tracrRNA.

Preferably, at least one CRISPR-protein complex comprising a CRISPR-nuclease and a guide RNA is used in the method of the invention. However, the skilled person straightforwardly understands that additional CRISPR-protein complexes can be used in the method of the invention, e.g. by the use of at least 2, 3, 4, 5, 6, 7, 8, 9, 10 or more different guide RNAs. These different guide RNAs van be designed to target and bind to the same sequence of interest. Alternatively, different guide RNAs may direct the CRISPR-protein complex to different genes of interest.

The transgene or mutation in a sequence of interest may be introduced prior to callus formation, during callus formation and/or after callus formation. The transgene or mutation is preferably introduced prior to the onset of shoot formation. Preferably, the transgene or mutation is present in at least a germline progenitor cell and/or clonally propagated tissue and/or plant part of a shoot formed in step (d) of the method of the invention. Preferably, the transgene or mutation is present in at least a germline progenitor cell and/or present in a cell giving rise to a clonally propagated tissue and/or plant part of a shoot selected in step (e). Preferably, the transgene or mutation is present in all germline progenitor cells and/or all clonally propagated tissues and/or plant parts of a shoot formed in step (d). Preferably, the transgene or mutation is present in at least one cell of the L2-shoot meristem layer of a shoot formed in step (d) of the method of the invention. Preferably, the transgene or mutation is present in all cells of the L2-shoot meristem layer of a shoot formed in step (d). The transgene or mutation may also be present in other cells, such as cells of the L1- and L3-shoot meristem layer. Optionally, all cells of a shoot formed in step (d) of the method of the invention comprise the transgene or mutation in a sequence of interest.

The transgene or mutation in a sequence of interest may be introduced in a cell of a plant from which the callus of step (a) is derived and/or in a cell of the plant from which the scion and/or rootstock of step (a) are excised. Preferably, the transgene or mutation in a sequence of interest is at least introduced in the cell of the callus of step (a) and/or in a cell of scion and/or rootstock of step (a).

The mutation may be introduced by transfecting the plant cell with a site-specific endonuclease, preferably a CRISPR-endonuclease. The transgene may be introduced by transfecting the plant cell with a transgene of interest. Transfection of a plant cell can be performed using any conventional means known to the person skilled in the art.

“Transfection” or “transformation” is understood herein as the delivery of a transgene and/or site-specific endonuclease protein or a nucleic acid molecule encoding the transgene and/or site-specific endonuclease into the plant cell. Said nucleic acid molecule may be DNA or RNA encoding said transgene and/or site-specific nuclease. Optionally the transgene and/or site-specific endonuclease is introduced by transfection of (pre-) mRNA. Transfection may further include the delivery of a guide RNA or a nucleic acid molecule encoding the guide RNA (to be) associated with a site-specific endonuclease into the plant cell. Optionally, the site-specific endonuclease is delivered as a CRISPR-endonuclease complex comprising a CRISPR-endonuclease complexed with a guide RNA. Alternatively or in addition, the CRISPR-endonuclease and the guide RNA are delivered into the plant cell, and form a complex intracellularly. Alternatively or in addition, the CRISPR-endonuclease is expressed from the transfected nucleic acid and forms intracellularly a complex with the, optionally expressed, guide RNA.

Preferably the transgene and/or site-specific endonuclease, or nucleic acid encoding the same, may be introduced as a protein, or in case of a CRISPR endonuclease as a protein-guide RNA complex (also called a ribonucleoprotein complex), into a cell of a recalcitrant plant using any conventional means known by the skilled person. Non-limiting examples of transfection include, but are not limited to, viral infection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo; protein transfection or nucleic acid transfection).

Transfection methods based upon the soil bacterium Agrobacterium tumefaciens may be particularly useful for introducing the nucleic acid molecule into a plant cell. Methods of co-culturing Agrobacterium with cultured plant cells or wounded tissue such as leaf tissue, root explants, hypocotyledons, stem pieces or tubers, for example, are well known in the art. See., e.g., Glick and Thompson, (eds.), Methods in Plant Molecular Biology and Biotechnology, Boca Raton, Fla.: CRC Press (1993). Microprojectile-mediated transformation also can be used to transfect the plant cell. This method, first described by Klein et al. (Nature 327:70-73 (1987)), relies on microprojectiles such as gold or tungsten that are coated with e.g. the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid encoding the transgene and/or the site-specific endonuclease, and optionally a (nucleic acid encoding) a guide RNA, may be introduced into a plant in a manner such that the nucleic acid is able to enter a plant cell(s), e.g., via an in vivo or ex vivo protocol. By “in vivo,” it is meant in the nucleic acid is administered to a living body of a plant e.g. infiltration. By “ex vivo” it is meant that cells or explants are modified outside of the plant, and then such cells or organs are regenerated into a shoot of a plant.

A number of vectors suitable for transformation of plant cells and/or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Examples include Agrobacterium tumefaciens-mediated transformation, as well as those methods e.g. disclosed by Herrera-Estrella et al. (1983) Nature 303:209, Bevan (1984) Nucl Acid Res. 12:8711-8721, Klee (1985) Bio/Technology 3:637-642. Conventional methods for transforming a plant cell include, but is not limited to, biolistic bombardment, polyethylene glycol transformation, and microinjection (see e.g. Danieli et al Nat. Biotechnol 16:345-348, 1998; Staub et al Nat. Biotechnol 18:333-338, 2000; O'Neill et al Plant J. 3:729-738, 1993; Knoblauch et al Nat. Biotechnol 17:906-909; U.S. Pat. Nos. 5,451,513, 5,545,817, 5,545,818, and 5,576,198; in Intl. Application No. WO 95/16783; and in Boynton et al., Methods in Enzymology 217:510-536 (1993), Svab et al., Proc. Natl. Acad. Sci. USA 90:913-917 (1993), and McBride et al., Proc. Natl. Acad. Sci. USA 91:7301-7305 (1994).

Preferably, the transgene is introduced in a cell of the callus of step (a), or plant giving rise to said callus, and/or the mutation is in a sequence of interest in a cell of the callus of step (a), or plant giving rise to said callus. The cell is preferably transfected with at least one of a transgene, a CRISPR endonuclease and/a one guide RNA. Preferably, the CRISPR-endonuclease and the guide RNA form a ribonucleoprotein complex that is transfected into the cell of the callus of step (a), plant giving rise to said callus. Preferably, said cell is a protoplast. Preferably the protoplast is transfected with a transgene protein and/or a CRISPR-guide RNA ribonucleoprotein complex using polyethylene glycol transformation, e.g. such as described in WO2017/222370 or WO2020/089448, which are incorporated herein by reference. The cell may be a cell in a single cell suspension, a protoplast, a cell present in a callus or a slice, and/or a cell present in a plant, preferably present in a graft union, preferably the graft union or junction of step (b) and (c) of the method provided herein.

Alternatively or in addition, a cell of callus, or of the plant giving rise to said callus, may be transfected with a nucleic acid molecule encoding the transgene and/or at least one site-specific endonuclease and/or at least one guide RNA. Optionally, said cells is a protoplast. Optionally, the protoplast is transfected with one or more plasmids encoding the transgene and/or CRISPR-endonuclease and a guide RNA using polyethylene glycol transformation, e.g. such as described in WO2018/115390 and WO/2020/011985, which are incorporated herein by reference.

Preferably the codon sequence of the transgene and/or site-specific endonuclease is optimized for expression in plant cells. The nucleic acid molecule encoding at least one transgene and/or site-specific endonuclease and/or at least one guide RNA is preferably comprised in a nucleic acid vector. The nucleic acid vector is preferably a vector for transient expression of the transgene and/or site-specific endonuclease and/or guide RNA. Alternatively, the nucleic acid vector is a vector for stable expression of the transgene and/or site-specific endonuclease and/or guide RNA. Optionally, at least one, optionally all, cell(s) of the callus of step a) of the method of the invention comprises a transgene integrated in its genome that encodes for a gene or interest and/or a programmable endonuclease, preferably for a CRISPR endonuclease, wherein said transgene and/or programmable endonuclease may be stably expressed or wherein the expression of said transgene and/or programmable endonuclease is under the control of an inducible or tissue specific promoter.

The transgene and/or site-specific endonuclease and optionally at least one guide RNA may thus be transcribed from an expression cassette comprised in the vector. The vector backbone may for example be a plasmid into which the expression cassette is integrated or, if a suitable transcription regulatory sequence is already present (for example a (inducible) promoter), only a desired nucleotide sequence (e.g. a sequence encoding the transgene and/or site-specific endonuclease) is integrated downstream of the transcription regulatory sequence.

The vector for use in the method of the invention may comprise further genetic elements to facilitate their use in molecular cloning, such as e.g. selectable markers, multiple cloning sites and the like. The vector backbone may for example be a binary or superbinary vector (see e.g. U.S. Pat. No. 5,591,616, US2002138879 and WO 95/06722), a co-integrate vector or a T-DNA vector, as known in the art.

Vectors for use in the method of the invention are preferably particularly suitable for introducing the expression of a transgene and/or site-specific endonuclease and optionally one or more guide RNAs into a plant cell, wherein the plant cell is preferably a plant cell of callus of step (a), or of the plant giving rise to said callus. A preferred expression vector is a naked DNA, a DNA complex or a viral vector.

A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, PEG, a dendrimer, an inorganic nanoparticle, a virosome and cell-penetrating peptides.

The vector for use in the method of the invention is preferably a viral expression vector. The viral vector van be an DNA virus or an RNA virus. The viral vector may be, or may be based on, a Tobamovirus, a Tobravirus, a Potexvirus, a Geminivirus, an Alfamovirus, a Cucumovirus, a Potyvirus, a Tombusvirus, a Hordeivirus, or a Nucleorhabdovirus.

The Tobamovirus viral vector may be at least one of a Tobacco Mosaic Virus (TMV) and a Sun Hemp Mosaic Virus (SHMV). The Tobravirus viral vector may be a Tobacco Rattle Virus (TRV). The Potex virus viral vector may be at least one of Potatovirus X (PVX) and the papaya mosaic potexvirus (PapMV). The Geminivirus viral vector may be a Comovirus Cowpea mosaic virus (CPMV). Further examples of suitable Geminivirus viral vectors may include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus. The Alfamovirus may an alfalfa mosaic virus (AMV). The Cucumovirus may be a cucumber mosaic virus (CMV). The Potyvirus may be a plum pox virus (PPV). The Tombusvirus may be a tomato bushy stunt virus (TBSV). The Hordeivirus may be a barley stripe mosaic virus. The Nucleorhabdovirus may be a Sonchus Yellow Net Virus (SYNV) (see e.g. Hefferon K, Plant Virus Expression Vectors: A Powerhouse for Global Health, Biomedicines. 2017, 5(3): 44 and Lico et al, Viral vectors for production of recombinant proteins in plants, J Cell Physiol, 2008; 216(2):366-77).

Preferably, the viral vector is selected from the group consisting of a Tobacco Rattle Virus (TRV), Tobacco Mosaic Virus (TMV), a Sonchus Yellow Net Virus (SYNV) and Potato Virus X (PVX). Preferably, the viral vector is at least one of a Tobacco Rattle Virus (TRV), a Tobacco Mosaic Virus (TMV) and a Sonchus Yellow Net Virus (SYNV).

The viral vector for use in the method of the invention may comprise a deletion of a gene to increase the packaging capacity of the virus. Preferably, the virus comprises a deletion of a gene encoding the coat protein (CP). A preferred viral vector comprising a deletion of the coat protein is a Tobamovirus virus or a Tobravirus virus. Preferably the viral vector comprising a deletion of a coat protein is a Tobamovirus, preferably the Tobacco Mosaic Virus (TMV). A preferred viral vector is the TMV RNA-based overexpression vector (TRBO), e.g. as described in Lindbo (TRBO: A High-Efficiency Tobacco Mosaic Virus RNA-Based Overexpression Vector, Plant Physiol, 2007; 145(4):1232-40). The viral vector may be a self-replicating RNA as e.g. described in WO2018/226972, which is incorporated herein by reference.

The vector, preferably the viral vector, may be comprised in an Agrobacterium to initially introduce the viral vector into a plant cell of the plant. After infection, the viral vector is expressed from the Agrobacterium in the plant cell. The viral vector may replicate and infect surrounding plant cells. The viral vector may be modified, e.g. by deletion of the coat protein, which prevents systemic spread of the virus.

The cell of callus of step (a), or of the plant giving rise to said callus, that is transfected will preferably develop into a tissue that is part of a newly formed shoot, wherein the tissue comprises one or more germline progenitor cells and/or one or more cells giving rise to a clonally propagated tissue and/or plant part. The transfected cell may be a primary transfected cell, or e.g. a secondary or subsequently transfected cell. As a non-limiting example, a cell of the callus of step (a), or plant giving rise to said callus, may be transfected with a vector, such as e.g. an agrobacterium and/or a viral vector, expressing a transgene and/or a site-specific endonuclease. The virus produced in these initially infected cells may spread and infect the (regenerated) callus, or the plant giving rise to the callus, i.e. in a secondary infection.

For example, one or more cells of the second plant may be infected with an agrobacterium comprising a viral vector expressing a transgene and/or a site-specific endonuclease. In the graft union, the produced virus may translocate to cells of the callus of step (a), or plant giving rise to said callus. Subsequent infection of the viral vector results in expression of a transgene and/or a site-specific endonuclease in one or more cells of the callus of step (a), or plant giving rise to said callus. The site-specific endonuclease will introduce a mutation in a sequence of interest in the said one or more cells and upon shoot formation, the mutation will be present in the formed shoots. The transgene may be integrated in the genome upon shoot formation and the transgene may be present in the formed shoots.

As indicated above, the method of the invention may further comprise a step (f) of growing a plant from the shoot selected in step (e). Optionally, especially in case root regeneration is cumbersome, step (f) may comprise a step of grafting the selected shoot on a graft compatible rootstock. The plant grown in step (f) preferably comprises at least one inflorescence for reproduction, i.e. to produce seed and/or progeny plants

More in particular, the invention provides for a method of generating a plant, wherein the method comprises the steps (a), (b), (c), (d), (e) and (f) as defined herein and further comprising the step of generating a plant from said shoot, wherein preferably said plant comprises at least one inflorescence. Optionally, the generated plant is free or substantially free of cells of, or derived from, the scion and/or rootstock of step (a). In other words, optionally the generated plant is a non-chimera plant having the same or substantially the same genotype of the callus of step (a) of the method of the invention. Hence, the generated plant may also be of the same species and variety as the callus of step (a). In case the method of the invention comprises the introduction of a mutation and/or transgene, preferably at least one of the cells, optionally all cells, of the generated plant also comprise(s) said mutation and/or transgene. Therefore, “substantially the same genotype” is to be understood herein as the same genotype albeit comprising a mutation and/or a transgene that may be introduced using a method of the invention.

In addition or alternatively, the germline cells, preferably the gametes, of the generated plant may have the same or substantially the same genotype as gametes of plant from which the callus of step (a) is derived, optionally comprising a mutation and/or transgene introduced in the cell of the callus of step (a) as further detailed herein. Optionally, the plant may be a chimeric plant that further comprises cells or tissue layers of, or derived of, the scion and/or rootstock of step (a). Optionally, said plant is used for producing seed and/or progeny by crossing, selfing and/or apomictic propagation in case of an apomictic genotype of the germline progenitor cells (i.e. apomictic reproduction). Optionally, the plant is pollinated and/or the pollen are used to pollinate another plant or the same plant (selfing). Optionally, said plant is used for producing a tissue and/or plant part for clonal propagation as defined herein, and optionally, said tissue and/or plant part is isolated and used clonal or vegetative propagation. Therefore, the invention also provides for a method of producing a plant or seed, comprising the steps (a), (b), (c), (d) and (e) as defined herein and further comprising the steps of generating a plant from the shoot selected in step (e) by vegetative or clonal propagation, wherein preferably said plant comprises at least one inflorescence; and optionally producing seed and/or a progeny plant of the generated plant by sexual or apomictic reproduction.

Hence, the method of the invention may be a method of producing a plant or seed, wherein said method comprises the steps of:
(a) intergrafting a callus between a scion and a rootstock;
(b) allowing graft junctions to be formed between the callus and each one of the scion and the rootstock to form a grafted union;
(c) generating a wound at or near at least one of the graft junctions;
(d) allowing the wounded grafted union to form shoots;
(e) selecting a shoot formed in step (d), wherein said shoot comprises cells derived from the callus of step (a);
(f) growing a plant from the selected shoot of step (e); and
(g) producing seed and/or progeny from the plant of step (f).

In case the method of the invention comprises the introduction of a mutation and/or transgene, the seed and/or progeny of the generated plant may be selected for having said mutation and/or transgene. The seed produced (or embryo of said seed) may have a genotype that is the same or substantially the same as offspring of the plant from which the callus of step (a) has been isolated or is part of, optionally with the exception of the introduced mutation and/or transgene.

Optionally, step (d) of regenerating a shoot comprises the formation of a callus prior to shoot regeneration. Hence, the method of the invention may be a method for producing a plant, wherein the plant comprises germline progenitor cells and/or a tissue and/or plant parts for clonal propagation, and wherein the method comprises the steps of:
(a) intergrafting a callus between a scion and a rootstock;
(b) allowing graft junctions to be formed between the callus and each one of the scion and the rootstock to form an grafted union;
(c) generating a wound at or near at least one of the graft junction;
(d) allowing the wounded grafted union to form shoot;
(e) selecting a shoot formed in step (d) comprising germline progenitor cells and/or cells giving rise to a tissue and/or plant parts for clonal propagation derived from the callus of step (a); and
(f) growing a plant from the selected shoot of step (e).

Optionally, multiple seeds and/or progeny plants are produced and the method further comprises a step of selecting at least one seed and/or progeny plant, preferably after genotyping and/or assessing the presence of the mutation and/or transgene that may have been introduced in at least one, or substantially all, cell(s) of the callus of step (a) of the method of the invention as detailed herein. The seed and/or progeny plant may be genotyped to assess whether the plant has the same or substantially the same genotype of the cell(s) of the callus of step (a). The seed may be allowed to germinate and develop into a plant.

Optionally, cells of the callus of step (a) of the method of the invention are cells with aberrant ploidy, and may be haploid. Hence the method of the invention may be a method to propagate haploid plant material. Said method may further comprise a step of screening plants and/or seeds for the production of callus of step (a) for ploidy levels.

Optionally, during step (b), (c) and/or (d) the genome may doubled spontaneously or may be doubled chemically, thereby generating at least one shoot that comprises or consists of doubled haploid cells. In that case, the genotype of the generated shoot may differ from the cells of the callus in step (a) in that the genome is doubled. Hence the method of the invention may be a method to produce doubled haploid plant material, and the method of the invention may comprise a step of screening regenerated plants and/or seeds for ploidy levels.

In another embodiment, the selected shoot of step (e) is not isolated but is allowed to grow an inflorescence on the shoot developed in step (d), (e) and optionally (f) of the method of the invention, wherein said shoot optionally comprises further shoots. Said inflorescence may be used for sexual or apomictic reproduction. Said inflorescence may be pollinated or pollen of said inflorescence is used to pollinate another plant or the same plant (i.e. the inflorescence is selfed).

As indicated herein, the method may further comprise a step of introducing in at least one, or substantially all, cell(s) of the callus of step (a) or in a cell originating therefrom in the shoot regenerated in step (d):
(i) a transgene; or
(ii) a mutation in a sequence of interest.

Preferably, said sequence of interest is an endogenous sequence of interest. In a method comprising the introduction of a transgene or mutation, preferably, the step of introducing the transgene or the mutation is prior to step (d), and even more preferably prior to step (a).

In addition or alternatively, in said method at least a germline progenitor cell and/or cells giving rise to clonally propagating tissue and/or plant part of the shoot regenerated in step (d) comprises the transgene or the mutation. Optionally, the mutation is introduced by programmed genome editing, preferably using a site-specific endonuclease, preferably a CRISPR endonuclease.

Optionally, the cells of the callus of step (a) of the method of the invention are (highly) heterogenetic, and the method of the invention is a method of propagating heterogenetic plant material. Optionally, the cells of the callus of step (a) are sterile, and the method of the invention is a method or propagating sterile plant material.

The plant may be grown from the shoot selected in step (d) of the method of the invention using any conventional culturing conditions known in the art by the skilled person. These culturing conditions may be dependent on the plant produced by the method of the invention and the skilled person knows how to adjust these conditions to generate an optimal environment for growing the plant produced by the method of the invention. The plant grown in step (f) may comprise a transgene or mutation in a sequence of interest as defined herein.

The method of the invention may further comprise a step (g) of producing or obtaining progeny of the plant grown in step (f). The progeny may e.g. be produced by sexual propagation, i.e. through the union of a pollen and an egg to produce a seed. Preferably, at least one of the pollen and the egg are derived from the plant produced in step (f). In case the method comprises the introduction of a transgene or mutation in a sequence of interest as defined herein, preferably, at least one of the pollen and the egg comprises the transgene or mutation in the sequence of interest. Optionally, both the pollen and egg are derived from the plant grown in step (f). Preferably, the pollen and the egg comprise the same transgene and/or mutation in the sequence of interest. Alternatively, the progeny is obtained by a-sexual (vegetative) propagation of the plant grown in step (f). Preferably, within such embodiment, the transgene and/or mutation in the sequence of interest is present in the tissue and/or plant part that is clonally propagated to form the next generation.

The invention also pertains to a plant obtainable by the method of the invention, preferably in step (f) by the method of the invention. The plant may be a chimera plant comprising cells having the same or substantially the same genotype of the callus of step (a) and cells or tissues having the same or substantially the same genotype of scion and/or rootstock of step (a). Preferably, the plant comprises germline or germline progenitor cells and/or a tissue and/or plant part for clonal propagation of the plant giving rise to the callus of step (a). Preferably, the plant comprises an L2-shoot meristem layer of said plant. Optionally the plant is a periclinal chimera and/or a plant comprising a transgene or mutation in a sequence of interest. Hence, the plant may be a non-natural plant, a man-made plant, a mutant plant and/or a transformed plant.

In an aspect, the invention thus concerns a periclinal chimera obtainable from the method of the invention, preferably obtainable from step (f) as defined herein. “Periclinal chimeras” are chimeras in which one or more entire cell (tissue) layer(s) L1, L2, and/or L3 is genetically distinct from another cell layer. In the case of periclinal chimeras, a single tissue layer itself is homogeneous and not chimeric. Periclinal chimeras are the most stable forms of chimeras, and produce distinctive and valuable plant phenotypes. These plants produce axillary buds that possess the same apical organization as the terminal meristem from which they were generated. Therefore, periclinal chimeras can be multiplied by vegetative propagation and maintain their chimera layer organization.

The periclinal chimera plant obtainable from the method of the invention preferably comprises at least one shoot meristem layer derived from the callus of step (a) and at least one shoot meristem layer derived from the scion and/or rootstock of step (a). Preferably at least one of the L1-, L2- and L3-shoot meristem layer is from a derived from the callus of step (a). The shoot meristem layer that is not from the callus op step (a) is preferably from a scion and/or rootstock of step (a). Preferably, the L2-shoot meristem layer of the periclinal chimera is of a the callus of step (a) and at least one of the L1- and L3-shoot meristem layer is of the second plant.

The L2-meristem layer and the L1- and L3-shoot meristem layer of the periclinal plant can be of the same or of a different genus. Preferably, the L2-meristem layer and the L1- and L3-shoot meristem layer of the periclinal plant are of the same genus. As a non-limiting example, the L1-, L2- and L3-shoot meristem layer can be of the genus Solanum or of the genus Capsicum. For example, the L2-shoot meristem layer can be from a Capsicum annuum plant and at least one of the L1- and L3-shoot meristem layer can be from a Capsicum baccatum plant. Similarly, the L2-shoot meristem layer can be from a Solanum tuberosum plant and at least one of the L1- and L3-shoot meristem layer can be from a Solanum lycopersicum plant.

The periclinal chimera may further comprise a transgene or a mutation in a sequence of interest. The mutation is preferably present in at least a germline or germline progenitor cell and/or a tissue and/or plant part for clonal propagation of the callus of step (a). Preferably, the transgene or mutation is in a cell located in at least one of the L1-, L2- and L3-shoot meristem layer of the periclinal chimera. Preferably, the transgene or mutation is present in a cell located in at least the L2-shoot meristem layer of the periclinal chimera.

A periclinal chimera produced by the method of the invention may find applications such as, but not limited to, specified in WO2018/115395 and/or WO2018/115396, which are incorporated herein by reference.

In a further aspect, the invention pertains to a plant obtainable from the method of the invention, wherein the plant comprises a transgene and/or mutation in a sequence of interest. Hence the plant may be a transgenic plant and/or mutant plant. The plant may be a man-made plant. Preferably, the transgene or mutation in the sequence of interest is located in germline or germline progenitor cells and/or tissue and/or plant part for clonal propagation of plant from which the callus of step (a) is derived. Therefore preferably, the plant comprises at least germline or germline progenitor cells of the callus of step (a) and/or a tissue and/or a plant part for clonal propagation of the callus of step (a) and preferably comprises the transgene or mutation in a sequence of interest. Preferably, the plant of the invention is not, or is not exclusively, obtained by an essentially biological process. The plant of the invention preferably differs at least from a plant occurring in nature, in that it contains at least one transgene or mutation in one sequence of interest. The transgene or mutation in the sequence of interest is preferably located in at least the germline or germline progenitor cells and/or tissue and/or plant part for clonal propagation of the plant. The transgene or mutation in the sequence of interest is preferably located in at least the L2-shoot meristem layer. The transgene or mutation in the sequence of interest is preferably present in at least one of the pollen and egg of the plant.

The plant preferably comprises at least germline or germline progenitor cells and/or tissues and/or plant parts for clonal propagation of derived from the callus of step (a). The plant preferably comprise at least the L2-shoot meristem layer derived from the callus of step (a). The plant obtainable from the method of the invention is preferably a plant having substantially the same genotype as the plant from which the callus of step (a) is derived, preferably comprising a transgene or mutation in a sequence of interest.

The invention further pertains to offspring or seed from the plant or periclinal chimera as defined herein. The offspring may be produced by sexual or a-sexual (vegetative) propagation. The offspring preferably comprises a transgene or mutation in a sequence of interest as defined herein. The integument of the seed may have a different genotype than the embryo. Preferably, the genotype of integument is from the scion and/or rootstock of step (a) and the genotype of the embryo is from the callus of step (a).

The invention also concerns a plant part or plant product derived from a plant obtained from the method of the invention, preferably of step (e), (f) or (g) of the method of the invention. Optionally, said plant part or plant product is characterized in that it comprises genetic material originating from both the callus as well as the rootstock and/or scion of step (a). Preferably, said plant part or plant product comprises cells or tissues or genetic material derived from the callus of step (a). Optionally, said plant part or plant product is free or substantially free of cells or tissues or genetic material that is derived from the scion and/or rootstock of step (a). Optionally, said plant part or plant product consist of cells or tissues or plant material are characterized in that it comprises the genotype of the callus of step (a). Optionally, the plant part or plant product is characterized in that it comprises a transgene or mutation in a sequence of interest. Such genetic material may be genomic DNA or fragments of genomic DNA. Such genetic material may be mitochondrial DNA or fragments of mitochondrial DNA. Such hereditary material may be chloroplast DNA or fragments of chloroplast DNA.

The plant part may be propagating or non-propagating material.

All patent and literature references cited in the present specification are hereby incorporated by reference in their entirety.

### FIGURE LEGENDS

FIG. 1. Schematic representation of the invention.

FIG. 2. (A) Purple leaf of the RUBY tomato marker line. (B) example of a 11 week old RUBY callus before grafting. (C) example of a grafted RUBY callus 7 days post grafting. (D) ungrafted control callus 2 weeks after cultivation (E) unsuccessful and (F) successful callus graft three weeks after grafting procedure.

FIG. 3. (A) Bright field image of a 2 weeks old callus graft. (B) Callus graft as in (A) minutes after root stock Acid Fuchsin (Red dye) application. Note the leaves have turned purple due to uptake of the acid fuchsin. (C) Bright field image of a longitudinal cross section through the stock-callus graft region. (D) Cross-section as in (C) showing chlorophyll and lignin auto-fluorescence as red and green signals, respectively. (E) Close-up of the indicated region in (D) revealing presence of spirally lignified callus cells. (F) Bright field image of a second independent 2 weeks old callus graft. (G) Callus graft as in (F) minutes after root stock CFDA (Green dye) application. Note the leaves have green veins due to uptake of the CFDA molecules. (H) Close-up of the stock-callus-scion tissues showing localized CFDA signal along the callus tissues (Green signal left of the red asterix). (I) Auto-florescent recording of (H) showing total tissue outline. (J) Shoot regeneration from scion tissues (shoots indicated by the red arrow).

FIG. 4. (A) Brightfield image of an incipient shoot regenerated from grafted RUBY marker callus tissue. (B) Auto-fluorescent recording of (A) revealing presence of phyllotactic pattern. (C) Same shoot 2 weeks after imaging of A and B. Green and red shoot were seedling and callus derived, respectively.

FIG. 5. (A) Twelve day old intergraft of protoplast derived Capsicum annuum c.v. Maor callus between a stock and scion of a C. baccatum seedling. Capsicum annuum c.v. Maor cells are marked by GFP expression. (B) leaf comprising differentiated cells of both the C. baccatum and Capsicum annuum c.v. Maor genotypes. (C) mericlinal chimeric shoot formed from the meristem associated with the leaf in (B). (D) Close up of the shoot apex indicated within the boxed area in (C) showing presence of GFP expressing Capsicum annuum c.v. Maor cells.

FIG. 6. (A) Intergraft of protoplast derived tomato cv. Moneyberg callus between a stock and scion of the F1 hybrid Solanum pennellii LA716 x Solanum lycopersicum LA3579. Protoplast derived Moneyberg cells are marked by GFP expression. (B) Sectorially chimeric leaf comprising cells of both the F1 hybrid and Moneyberg genotypes. (C) Chimeric shoot formed from the mericlinal meristem from which the leaf in (B) was derived.

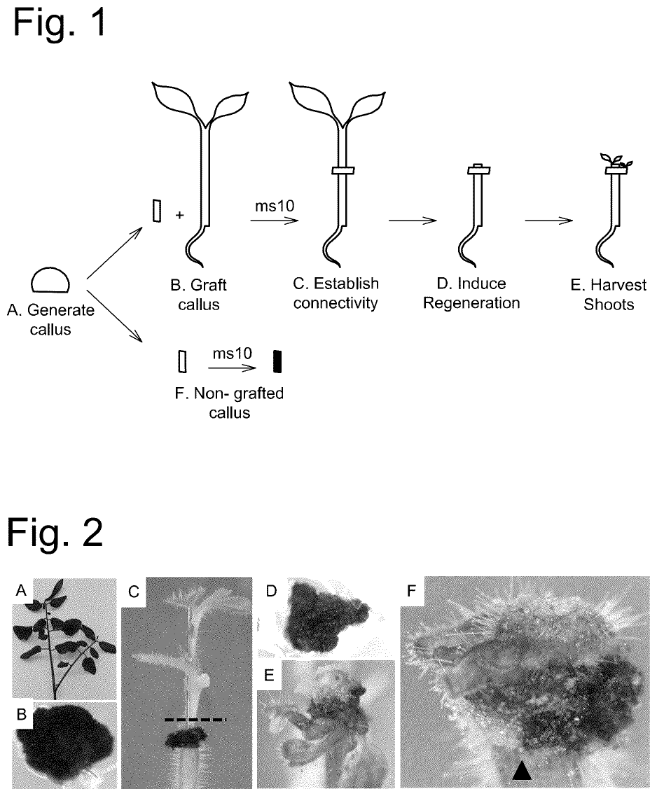

### EXAMPLE 1

Protoplast from leaves of Solanum lycopersicum c.v. Garden Pearl stably transformed to express purple RUBY marker (He et al. Horticulture research 2020, 7 (1): 152 doi: 10.1038/s41438-020-00390-1) were cultivated on callus induction medium for 11 weeks to generate purple calli (FIG. 2A,B). RUBY calli were inter-grafted between the stock and scion of a highly regenerative tomato genotype, i.e. an F1 hybrid of Solanum pennellii accession LA716 and Solanum lycopersicum accession LA3579 containing the semi-dominant marker xa (FIG. 2C). The semi-dominant phenotypic marker xa in heterozygous condition, causes yellow leaves when present in L2 and/or L3 (Szymkowiak and Sussex. Plant Cell 1992, 4:1089-1100). In successful grafts, callus tissues remained viable as indicated by the presence of the RUBY color and formation of a continuous callus mass within the graft junction (FIG. 2F). In contrast, non-grafted control calli died after two weeks of cultivation on hormone free medium (FIG. 2D). Similar responses were observed in unsuccessful grafting attempts, wherein callus tissues rapidly browned and shriveled (FIG. 2E).

Successfully grafted callus tissues remained viable for over 7 weeks when cultured on a near vertical plate with 25 mL MS10 agar. This suggests formation of stock/scion connectivity as it is critically required for stock-scion water/nutrient exchange. Three lines of evidence collectively suggest that a certain level of connectivity becomes established after the applied grafting procedure.

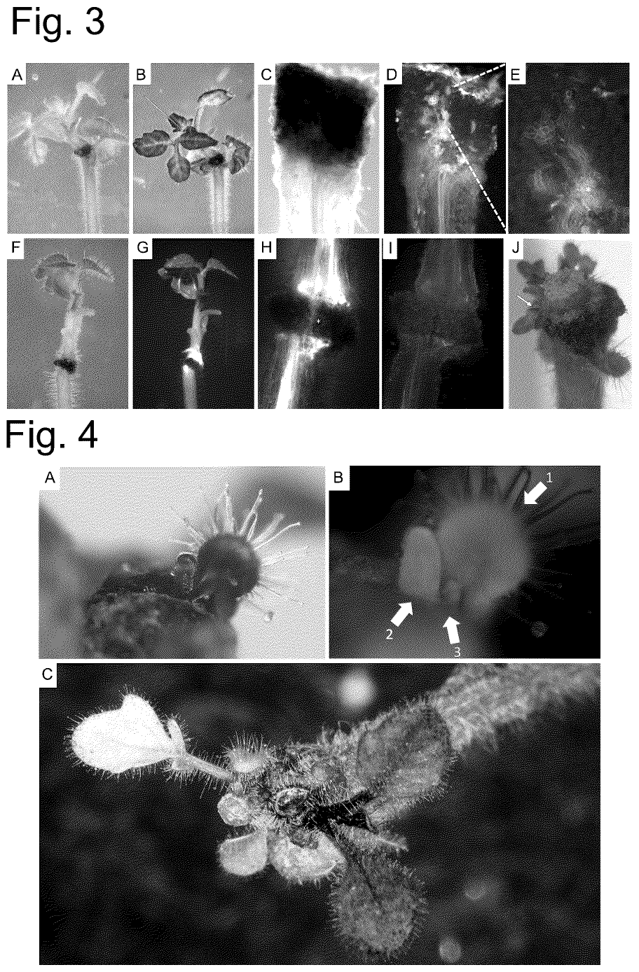

First, Acid Fuchsin solution rapidly accumulated in the scion when applied to the stock root system (FIG. 3A,B). In line with presence of a conductive callus tissue type, spirally lignified callus cells were observed in the callus grafted between the stock and scion (FIG. 3 C-E). Spiral lignin depositioning is a unique hallmark of vascular xylem cells.

Second, similar results were obtained when the experiment was repeated with the cell-permeable, amine-reactive green fluorophore 5-(and-6)-carboxyfluorescein diacetate (CFDA) as the tracer molecule (FIG. 3F,G). Strikingly, the pattern of CFDA within the callus tissue was localized to a central region of the grafted callus tissue (FIG. 3H,I).

Third, shoot regeneration was observed from scion tissues indicating prolonged scion tissue viability (see below and FIG. 2F, FIG. 3J). Hence, the grafted callus tissue established stock/scion connectivity.

At two weeks after grafting, callus grafts were decapitated at the callus-scion interface to induce naturally occurring tomato shoot regeneration (FIG. 2C, cut site as indicated by the dotted red line). Decapitated grafts were transferred to the greenhouse where stock/scion tissues naturally regenerated shoots under ex-vitro conditions (FIG. 2F,3J). Strikingly, callus grafted tissues formed organs in parallel to regeneration of shoots (FIG. 4A,B). Importantly, the first observed organ was capable to develop into a functional shoot (FIG. 4C). This proofs effective regeneration upon callus grafting.

### EXAMPLE 2

Maor pepper (Capsicum annuum) is known for being a recalcitrant plant, showing no regeneration with any conventional tissue or protoplast culture regeneration procedure. A stable transgenic Capsicum annuum c.v. Maor plant was generated by transformation with the construct pKG11052, as described previously (Example 2 of WO 2019/211296). The construct pKG11052 comprises the following promoter-transgene expression cassettes:
CaMV 35S—XVE
CaMV 35S—GVG
XVE inducible promoter—WIND1
GVG inducible promoter—PLT1
GVG inducible promoter—WOX5
CaMV 35S—erGFP

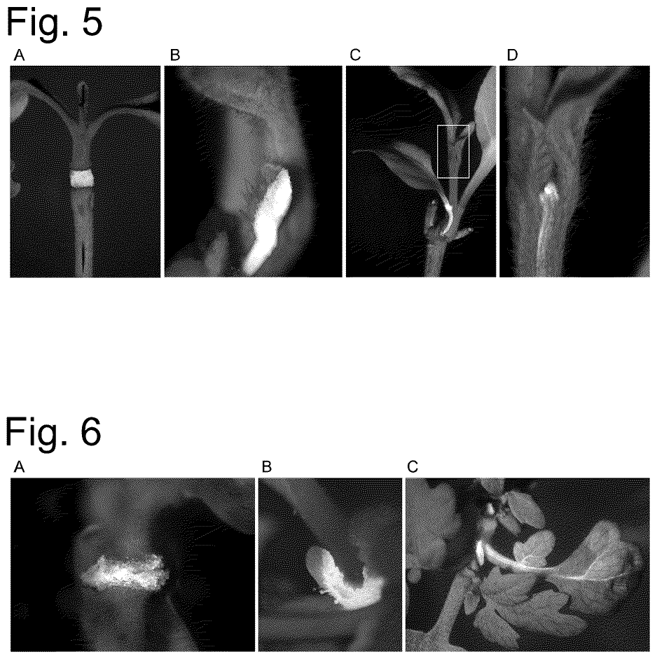

Prototoplasts from stably transfected GFP marker-expressing pepper cv Maor leaves were cultivated on callus induction medium for 7 weeks to generated calli. Maor GFP-expressing calli were intergrafted between stocks and scions of 9 day old wild type C. baccatum seedlings (FIG. 5A). Wound healing was allowed for 12 days (cultured on a near vertical plate with 25 mL MS10 agar) after which the inter grafts were decapitated at the callus-scion graft junction and were kept on the near vertical plates with 25 mL MS10 agar. The C. baccatum genotype spontaneous regeneration at the decapitation surface without the addition of any hormones. Two weeks after decapitation a mericlinal leaf had formed comprising differentiated cells of both the C. baccatum and Capsicum annuum c.v. Maor genotypes (FIG. 5B). The chimeric nature was maintained during developmental growth which produced a shoot including Capsicum annuum c.v. Maor cells containing shoot meristems (FIG. 5C, D).

### EXAMPLE 3

A stable transgenic tomato cv. Moneyberg plant comprising the construct pKG11052 (as described in Example 2) was generated. Protoplasts from GFP marker expressing leaves were cultivated on callus induction medium for 6 weeks to generate calli. GFP expressing calli were intergrafted between stocks and scions of a highly regenerative tomato genotype, i.e. the F1 hybrid Solanum lycopersicum LA3579 x Solanum pennellii LA716 (FIG. 6A). Graft healing was allowed for 10 days (cultured on a near vertical plate with 25 mL MS10 agar) after which the inter grafts were decapitated at the callus-scion graft junction. The F1 hybrid genotype regenerated spontaneously from the decapitation surface in the absence of hormone application and cultured on a near vertical plate with 25 mL MS10 agar. Two weeks after decapitation, a sectorial chimeric leaf had formed comprising differentiated cells of both the tomato F1 hybrid and protoplast derived cv. Moneyberg genotypes (FIG. 6B). The with this leaf associated mericlinal meristem produced a chimeric shoot and shoot meristems that comprised protoplast derived cells of the tomato cv. Moneyberg genotype (FIG. 6C).

1. Method of generating and selecting a shoot of a plant, wherein the method comprises the steps of:
(a) intergrafting a callus between a scion and a rootstock;
(b) allowing graft junctions to be formed between the callus and each one of the scion and the rootstock, to form a grafted union;
(c) generating a wound at or near at least one of the graft junctions;
(d) allowing the wounded grafted union to form shoots;
(e) selecting a shoot formed in step (d), wherein said shoot comprises cells derived from the callus of step (a); and optionally
(f) growing a plant from the selected shoot of step (e).

2. Method according to claim 1, wherein in step (e) the selected shoot comprises a germline progenitor cell derived from the callus of step (a) and wherein optionally the method further comprises step (f) and a step (g) of obtaining seed or progeny of the plant grown in step (f).

3. Method according to claim 1, wherein the method further comprises step (f) and a step (g) of obtaining progeny of the plant grown in step (f) by vegetative propagation.

4. Method according to claim 1, wherein the callus in step (a) is of a first plant and the scion and/or rootstock are of a second plant.

5. Method according to claim 4, wherein the scion and the rootstock are of the same or a similar plant.

6. Method according to claim 1, wherein the wounding of step (c) is removal of the shoot apical meristem by decapitation.

7. Method according to claim 1, wherein in step (d) the axis of the wounded grafted union is substantially perpendicular to the earth surface, and wherein the root apical meristem is closer to the earth surface as compared to the shoot apical meristem.

8. Method according to claim 1, wherein step (d) of allowing shoot formation comprises the steps of:
d1) allowing callus to be formed at or near the graft junction; and
d2) allowing a shoot to grow from said callus.

9. Method according to claim 1, wherein the method further comprises prior to step (a) a step of growing the callus of step (a) from a protoplast.

10. Method according to claim 9, wherein the method further comprises a step of introducing into the protoplast a transgene and/or a mutation in a sequence of interest, and wherein in step (e) the selected shoot comprises a germline progenitor cell, or a germline cell derived therefrom, comprising the transgene and/or the mutation.

11. Method according to claim 1, wherein the method further comprises a step of introducing into a cell located in the callus of step (a) and/or in the shoot formed in step (d) a transgene and/or a mutation in a sequence of interest, and wherein in step (e) the selected shoot comprises a germline progenitor cell, or a germline cell derived therefrom, comprising the transgene and/or the mutation.

12. Method according to claim 10, wherein the method comprises step (f) and wherein a plant part of the plant grown in step (f) comprises the transgene and/or the mutation.

13. Method according claim 10, wherein the mutation is introduced by programmed genome editing.

14. Plant obtainable by the method according to claim 10, wherein said plant comprises at least one of:
i) a germline progenitor cell and/or a germline cell derived therefrom, of the callus of step (a); and
ii) a plant part for vegetative propagation of the callus of step (a),
wherein the germline progenitor cell, germline cell and/or a plant part comprises the transgene and/or the mutation.

15. Plant according to claim 14, wherein said plant comprises cells derived from the callus of step (a) and cells derived from the scion and/or rootstock of step (a).


SEQUENCE LISTING (12 sequences)

SEQ ID NO: 1 - protein, 182 aa, Arabidopsis thaliana
MSFSVKGRSLRGNNNGGTGTKCGRWNPTVEQLKILTDLFRAGLRTPTTDQIQKISTELSFYGKIESKNVFYWFQNHKARERQKRRKISIDFDHHHHQPSTRDVFEISEEDCQEEEKVIETLQLFPVNSFEDSNSKVDKMRARGNNQYREYIRETTTTSFSPYSSCGAEMEHPPPLDLRLSFL

SEQ ID NO: 2 - protein, 574 aa, Arabidopsis thaliana
MNSNNWLGFPLSPNNSSLPPHEYNLGLVSDHMDNPFQTQEWNMINPHGGGGDEGGEVPKVADFLGVSKPDENQSNHLVAYNDSDYYFHTNSLMPSVQSNDVVVAACDSNTPNNSSYHELQESAHNLQSLTLSMGTTAGNNVVDKASPSETTGDNASGGALAVVETATPRRALDTFGQRTSIYRGVTRHRWTGRYEAHLWDNSCRREGQSRKGRQVYLGGYDKEDKAARSYDLAALKYWGPSTTTNFPITNYEKEVEEMKHMTRQEFVAAIRRKSSGFSRGASMYRGVTRHHQHGRWQARIGRVAGNKDLYLGTFSTEEEAAEAYDIAAIKFRGLNAVTNFEINRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 3 - protein, 334 aa, Arabidopsis thaliana
MAAAMNLYTCSRSFQDSGGELMDALVPFIKSVSDSPSSSSAASASAFLHPSAFSLPPLPGYYPDSTFLTQPFSYGSDLQQTGSLIGLNNLSSSQIHQIQSQIHHPLPPTHHNNNNSFSNLLSPKPLLMKQSGVAGSCFAYGSGVPSKPTKLYRGVRQRHWGKWVAEIRLPRNRTRLWLGTFDTAEEAALAYDKAAYKLRGDFARLNFPNLRHNGSHIGGDFGEYKPLHSSVDAKLEAICKSMAETQKQDKSTKSSKKREKKVSSPDLSEKVKAEENSVSIGGSPPVTEFEESTAGSSPLSDLTFADPEEPPQWNETFSLEKYPSYEIDWDSILA

SEQ ID NO: 4 - protein, 14 aa, synthetic construct
MEDPTMAPKKKRKV

SEQ ID NO: 5 - protein, 16 aa, synthetic construct
KRPAATKKAGQAKKKK

SEQ ID NO: 6 - protein, 1368 aa, synthetic construct [sequence omitted]

SEQ ID NO: 7 - DNA, 4104 bp, synthetic construct
  source 1..4104: mol_type = other DNA
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 8 - protein, 1082 aa, synthetic construct
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 9 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 10 - DNA, 3900 bp, synthetic construct
  source 1..3900: mol_type = other DNA
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 11 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 12 - protein, 1274 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV